SEQUENCE LISTING (33 sequences)

SEQ ID NO: 1 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPMTYILKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECINLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 1270 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLIESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 1268 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTCVIAWNSNNLDSKVGGNYNYLYRLFRKSNLKPFERDISTEIYQAGSTPNGVEGFNCYFPLQSYGFQPTNGVGYQPYRVVVLSFELLHAPATVCGPKKSTNLVKNKCVNFNFNGLTGTGVLIESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 1273 aa, Severe acute respiratory syndrome coronavirus 2
MFVFLVLLPLVSSQCVNLTTRTQLPPAYTNSFTRRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPIYLVRDLPQGFSALEPLVDLPIGINITRFQTLLAHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1274 aa, Severe acute respiratory syndrome coronavirus 2
MFVFLVLLPLVSSQCVNLTTRTQLPPAYTNSFTRRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPIYLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - protein, 9 aa, synthetic construct
CNGVEGFNC

SEQ ID NO: 7 - protein, 11 aa, synthetic construct
YGFQPTNGVGY

SEQ ID NO: 8 - protein, 9 aa, synthetic construct
CNGVKGFNC

SEQ ID NO: 9 - protein, 11 aa, synthetic construct
YGFQPTYGVGY

SEQ ID NO: 10 - protein, 9 aa, synthetic construct
CNGVQGFNC

SEQ ID NO: 11 - protein, 9 aa, synthetic construct
CNGVRGFNC

SEQ ID NO: 12 - protein, 8 aa, synthetic construct
CNGVNFNC

SEQ ID NO: 13 - protein, 9 aa, synthetic construct
CNGVHGFNC

SEQ ID NO: 14 - protein, 9 aa, synthetic construct
CNGVPGFNC

SEQ ID NO: 15 - protein, 9 aa, synthetic construct
CNGVSGFNC

SEQ ID NO: 16 - protein, 9 aa, synthetic construct
CNGVLGFNC

SEQ ID NO: 17 - protein, 11 aa, synthetic construct
YGFQPTKGVGY

SEQ ID NO: 18 - protein, 11 aa, synthetic construct
YGFQPTRGVGY

SEQ ID NO: 19 - protein, 11 aa, synthetic construct
YGFQPTHGVGY

SEQ ID NO: 20 - protein, 11 aa, synthetic construct
YGFQPTEGVGY

SEQ ID NO: 21 - protein, 11 aa, synthetic construct
YGFQPTSGVGY

SEQ ID NO: 22 - protein, 11 aa, synthetic construct
YGFQPTGGVGY

SEQ ID NO: 23 - protein, 11 aa, synthetic construct
YGFQPTIGVGY

SEQ ID NO: 24 - protein, 11 aa, synthetic construct
YGFQPTFGVGY

SEQ ID NO: 25 - protein, 11 aa, synthetic construct
YGFQPTDGVGY

SEQ ID NO: 26 - protein, 11 aa, synthetic construct
YGFQPTCGVGY

SEQ ID NO: 27 - protein, 12 aa, synthetic construct
YNYRYRLFRKSN

SEQ ID NO: 28 - protein, 10 aa, synthetic construct
CDIPIQAGIC

SEQ ID NO: 29 - protein, 10 aa, synthetic construct
CDIPIHAGIC

SEQ ID NO: 30 - protein, 10 aa, synthetic construct
NSPRRARSVA

SEQ ID NO: 31 - protein, 10 aa, synthetic construct
NSHRRARSVA

SEQ ID NO: 32 - protein, 11 aa, synthetic construct
IAWNSNNLDSK

SEQ ID NO: 33 - protein, 11 aa, synthetic construct
IAWNSNKLDSK

(19) United States
(12) Patent Application Publication
(10) Pub. No.: US 2023/0270675 A1
(43) Pub. Date: Aug. 31, 2023

(54) ADJUVANTED VACCINE COMPOSITION AND METHODS

(71) Applicant: D4 Labs, LLC, Pacific Grove, CA (US)

(72) Inventors: Michael Bowe, Marina, CA (US); David Craig Wright, Pacific Grove, CA (US)

(21) Appl. No.: 17/987,057

(22) Filed: Nov. 15, 2022

Related U.S. Application Data
(63) Continuation of application No. PCT/US2022/025622, filed on Apr. 20, 2022.
(60) Provisional application No. 63/177,085, filed on Apr. 20, 2021.

(51) Int. Cl. A61K 9/127; A61K 39/215; A61K 39/39; A61P 31/14

(52) U.S. Cl. CPC A61K 9/1272; A61K 39/215; A61K 39/39; A61P 31/14; A61K 2039/55555

(57) ABSTRACT

Disclosed herein are immunogenic compositions (e.g., vaccines) and methods of using and preparing the same. In some embodiments, the immunogenic compositions are suitable for use in treating or preventing an infectious disease, such as SARS-CoV-2 or HIV.

### CROSS-REFERENCE TO RELATED APPLICATION

This application claims priority to U.S. Provisional Application 63/177,085, filed Apr. 20, 2021, which is incorporated herein by reference in its entirety.

### SEQUENCE LISTING

The instant application contains a Sequence Listing which has been submitted electronically in XML file format and is hereby incorporated by reference in its entirety. Said XML copy, created on May 16, 2023, is named 31579_105001_US_SL.xml and is 36,051 bytes in size.

### FIELD OF THE INVENTION

Disclosed herein are adjuvanted peptide vaccines and methods for preparing and using the same. In particular, adjuvanted SARS-COV-2 and HIV vaccines are disclosed.

Advantageously, the adjuvanted peptides vaccines disclosed herein do not utilize squalene.

### BACKGROUND OF THE INVENTION

Vaccine development and usage over the years has significantly reduced the number of infections and diseases on a global basis. The need for vaccines persists, however, including for the treatment of emerging viral threats (e.g., SARS-COV-2) and viral agents that have eluded successful vaccine strategies (e.g., HIV).

Traditionally, vaccines were based on the use of an intact viral agent, either inactivated or live attenuated. In more recent years, vaccines have utilized subunits of the viral agent, including naturally occurring immunogenic polypeptide(s) or synthetic peptides that correspond to highly conserved regions required for viral function. These subunit vaccines are sufficient for activation of the appropriate cellular and humoral responses, while eliminating allergenic and/or reactogenic responses.

Peptide vaccines, in particular, offer numerous advantages over traditional vaccines including cost and stability. However, challenges remain to their widespread clinical use including weak immunogenicity. Immunostimulatory adjuvants have been used to enhance the immune response to peptide vaccines, but many traditional agents (including agents used to adjuvant polypeptides) have proven ineffective for peptide vaccines.

There remains a need for novel vaccine strategies, including novel peptide vaccine strategies, particularly for emerging and recalcitrant viral diseases.

### SUMMARY OF THE INVENTION

Disclosed herein are adjuvanted peptide vaccines and methods for preparing and using the same.

In a first aspect, an adjuvanted peptide vaccine is disclosed comprising at least one synthetic peptide and a liposome, wherein the liposome is a non-phospholipid liposome incorporating Vitamin E and wherein the at least one synthetic peptide is mixed with or encapsulated within the liposome.

In one embodiment, the adjuvanted peptide vaccine comprises two or more linear synthetic peptides. In certain embodiments, the two or more linear peptides hare the same amino acid sequence. In other embodiments, the two or more linear peptides differ by amino acid sequence, i.e., the linear peptides are mixed. In certain embodiments, the adjuvanted peptide vaccine generates antibodies that recognize the RBD and S1 proteins of SARS-CoV-2.

In another embodiment, the adjuvanted peptide vaccine comprises a multimeric synthetic peptide comprising at least two peptides. In certain embodiments, the multimeric peptide is branched. In certain embodiments, the multimeric peptide is homomeric. In other embodiments, the multimeric peptide is heteromeric. In certain embodiments, the adjuvanted peptide vaccine cross-reacts with the RBD and S1 protein of SARS-CoV-2 In a particular embodiment, the at least one synthetic peptide is derived from a viral protein, and more particularly a viral protein of SARS-CoV-2 or HIV.

In a particular embodiment, the at least one synthetic peptide is derived from the spike (S) protein of SARS-CoV-2 and more particularly, the Receptor Binding Motif (RBM) of the S protein.

In one embodiment, the adjuvanted peptide vaccine comprises two or more linear synthetic peptides derived from SEQ ID NO: 1 or a variant or homolog thereof, for example, a variant comprising one or more substitution mutations in the amino acid sequence of the peptide(s).

In one embodiment, the adjuvanted peptide vaccine comprises at least one multimeric peptide comprising two or more peptides derived from SEQ ID. NO: 1 or a variant or homolog thereof, for example, a variant comprising one or more substitution mutations. In certain embodiments, the at least one multimeric peptide is homomeric. In other embodiments, the at least one multimeric peptide is heteromeric.

In certain embodiments, the adjuvanted peptide vaccine comprises two or more linear peptides comprising amino acids 480-488 and 495-505 of SEQ ID NO: 1, or variants or homologs thereof, e.g., a variant comprising one or more substitution mutations.

In a particular embodiment, the adjuvanted peptide vaccine comprises two or more linear peptides selected from the group consisting of cngvegfnc (SEQ ID NO: 6), ygfqptngvgy (SEQ ID NO: 7), cngvKgfnc (SEQ ID NO: 8), ygfqptYgvgy (SEQ ID NO: 9) and combinations thereof.

In certain embodiments, the adjuvanted peptide vaccine comprises a multimeric peptide comprising at least two peptides comprising amino acids 480-488 and 495-505 of SEQ ID NO: 1, or variants or homologs thereof, e.g., a variant comprising one or more substitution mutations.

In certain embodiments, the multimeric peptide is a septamer comprising the peptides cngvegfnc (SEQ ID NO: 6), ygfqptngvgy (SEQ ID NO: 7), cngvKgfnc (SEQ ID NO: 8), ygfqptYgvgy (SEQ ID NO: 9) or combinations thereof. In certain embodiments, the septamer cross-reacts with the modified RBD and modified S1 protein of SARS-CoV-2.

In one embodiment, the liposome comprises a lipid bilayer comprising one or more non-ionic surfactants and optionally, a helper lipid (e.g., cholesterol).

In a particular embodiment, the lipid bilayer comprises between two (2) and ten (10) bilayers surrounding an amorphous central cavity. In certain embodiments, the lipid bilayer incorporates vitamin E. In one embodiment, the central cavity of the liposome comprises vitamin E.

In a second aspect, a method is disclosed for generating an immune response comprising administering the adjuvanted peptide vaccine disclosed herein to a subject, thereby generating an immune response.

In a third aspect, a method is disclosed for preventing an infection in a subject in need thereof comprising administering the adjuvanted peptide vaccine disclosed herein to the subject, thereby preventing the infection, i.e., conferring protective immunity.

In certain embodiments, the adjuvanted peptides vaccine disclosed herein has one or more improved properties compared to the at least one synthetic peptide in the absence of the liposome, including enhanced antigen immunogenicity, binding affinity, cytotoxic potency, and/or selectivity.

In certain embodiments, the adjuvanted peptide vaccines disclosed herein increase the immune response to the peptide vaccine to a greater degree than known adjuvants such as Freund's complete adjuvant, alum, and aluminum hydroxides.

In certain embodiments, the adjuvanted peptide vaccine is administered in two or more doses.

In certain embodiments, the adjuvanted peptide vaccine is administered intramuscularly, or subcutaneously.

In certain embodiments, the adjuvanted peptide vaccine is administered in combination with one or more additional therapeutic agents.

### BRIEF DESCRIPTION OF THE FIGURES

FIG. 1: Depicts blot images for IgG antibodies directed against the Receptor Binding Domain and the S1 subunit of the Spike protein. The animals were immunized with SVE-Peptide A cngvegfnc (SEQ ID NO: 6) subcutaneously. RBD DSO and S1 DSO are the negative blots which are not red. RBD-SQ, and S1-SQ blots are red and positive for antibodies to both proteins Receptor Binding Domain and S1. Antibodies to the Receptor Binding Domain are a surrogate for neutralizing antibodies to SARS-CoV-2. The dilution of the sera was 1:20.

FIG. 2. Depicts blot images for IgG antibodies directed against the proteins Receptor Binding Domain and the S1 subunit of the Spike protein. The animals were immunized subcutaneously with SVE-Peptide D construct which contains four copies of cngvegfnc (SEQ ID NO: 6) and three copies of ygfqptngvgy (SEQ ID NO: 7) in a lysine backbone structure. RBD DSO and S1 DSO are the negative blots which are not red. RBD-SQ, and S1-SQ blots are red and positive for antibodies to both proteins Receptor Binding Domain and S1. Antibodies to the Receptor Binding Domain are a surrogate for neutralizing antibodies to SARS-CoV-2. The dilution of the sera was 1:20.

FIG. 3(A)-(B). Depict exemplary embodiments of a synthetic peptide(s) that can be used in the vaccines disclosed herein. Figure discloses SEQ ID NOS 6, 8, 10, 7, 9 and 11-33, respectively, in order of appearance.

### DETAILED DESCRIPTION

### I. Definitions

The term “about” as used herein refers to a value or element that is similar to a stated reference value or element. In certain embodiments, the term “about” or “approximately” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

The term “adjuvant” as used herein refers to a substance whose admixture with an administered immunogenic determinant/antigen construct increases or otherwise modifies the immune response to said determinant. Immunological adjuvants function by attracting macrophages to the antigen and then to presenting said antigens to the regional lymph nodes and initiating an effective antigenic response. Conventional adjuvants can serve as vehicles for the antigen, and as nonspecific immunological stimulants. In one embodiment, the liposome (e.g., the paucimellar liposome) serves as an adjuvant for the peptide vaccine disclosed herein and in certain embodiments, incorporates Vitamin E.

The term “administering” as used herein means either directly administering a compound or composition of the present invention. Any route of administration, such as topical, subcutaneous, peritoneal, intravenous, intraarterial, inhalation, vaginal, rectal, nasal, buccal, introduction into the cerebrospinal fluid, or instillation into body compartments can be used. The terms and phrases “administering” and “administration of,” when used in connection with a compound or pharmaceutical composition (and grammatical equivalents) refer both to direct administration, which may be administration to a patient by a medical professional or by self-administration by the patient, and/or to indirect administration, which may be the act of prescribing a drug.

The term “affinity” as used herein refers to the equilibrium constant for the reversible binding of two agents and is expressed as a dissociation constant (K_{D}).

The term “amino acid” or “amino acids” as used herein is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids (stereoisomers). Abbreviations for amino acids are well understood in the art.

The terms “amino-terminal” and “carboxyl-terminal” are used herein to denote positions within polypeptides. Where the context allows, these terms are used with reference to a particular sequence or portion of a peptide to denote proximity or relative position.

The term “amphiphilic” as used herein refers means exhibiting characteristics of hydrophilicity and lipophilicity. Common amphiphilic substances are soaps, detergents and lipoproteins. Other examples of amphiphilic compounds are: Saponins, phospholipids, glycolipids, polysorbates.

The term “antigen” as used herein refers to a molecule with one or more epitopes that stimulate a host's immune system to make a secretory, humoral and/or cellular antigen-specific response, or to a DNA molecule that is capable of producing such an antigen in a vertebrate. The term is also used interchangeably with “immunogen.” For example, a specific antigen can be complete protein, portions of a protein, peptides, fusion proteins, glycosylated proteins and combinations thereof.

The term “binding” as used herein refers to direct association between two molecules, due to, for example, covalent, electrostatic, hydrophobic, and ionic and/or hydrogen-bond interactions, including interactions such as salt bridges and water bridges. “Specific binding” refers to binding with an affinity of at least about 10⁻⁷ M or greater.

The term “boost” as used herein refers to the administration of an additional dose of an immunizing agent, such as a vaccine, administered at a time after the initial dose to sustain the immune response elicited by the previous dose of the same agent. In certain embodiments, the immunogenic composition disclosed herein is a booster vaccine.

The term “carrier” as used herein includes any solvent(s), dispersion medium, coating(s), diluent(s), buffer(s), isotonic agent(s), solution(s), suspension(s), colloid(s), inert (s), or such like, or a combination thereof that is pharmaceutically acceptable for administration to the relevant animal or acceptable for a therapeutic or diagnostic purpose, as applicable.

The term “cholesterol derivative” as used herein refers to a derivative of the molecule cholesterol. Representative, non-limiting examples of cholesterol derivates include ldosterone, beclomethasone, betamethasone, cholesterol, cloprednol, cortisone, cortivazol, deoxycortone, desonide, dexamethasone, difluorocortolone, fluclorolone, fluorocortisone, flumethasone, flunisolide, fluocinolone, fluocinonide, fluorocortolone, fluorometholone, flurandrenolone, halcinonide, hydrocortisone, meprednisone, methylprednisolone, oxandrolone, oxymetholone, paramethasone, prednisolone, prednisone, stanozolol, and triamicinolone, testosterone, dehvdroeniandrosterone, androstenedione, dihydrotestosterone, aldosterone, estradiol, estrone, estriol, cortisol, oroaesterone and hydroxycholesterol.

The term “combination” as used herein means an assemblage of agents for use in therapy either by simultaneous or separate (e.g., sequential or concomitant) administration. In certain embodiments, the immunogenic composition is administered to the subject in combination with one or more additional therapeutic agents (e.g., small molecule therapeutics, biologics).

The terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The term “conservative amino acid substitution” as used herein refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

The term “cross-reacts” as used herein refers to the reaction between an antigen and an antibody that was generated against a different but similar antigen.

The term “encapsulate” as used herein refers the lipid vesicle forming an impediment to free diffusion into solution by an association with or around an agent of interest, e.g., a lipid vesicle may encapsulate an agent within a lipid layer or within an aqueous compartment inside or between lipid layers.

The term “homologous” as used herein refers to the subunit sequence similarity between two polymeric molecules, e.g., between two polypeptide molecules. When a subunit position in both of the two molecules is occupied by the same monomeric subunit, e.g., amino acid, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology.

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and)(BLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=1 1 to obtain nucleotide sequences homologous to a nucleic acid described herein. BLAST protein searches can be performed with the)(BLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The term “homomeric” as used herein refers to something composed of one repeating subunit. This is in contrast to “heteromeric”, which refers to something (e.g., a peptide) composed of different subunits.

The term “identical” or percent “identity,” in the context of two or more nucleic acids or polypeptide sequences may refer to two or more sequences or subsequences that are the same or have a specified percentage of amino acid residues or nucleotides that are the same. In some cases, 2 or more sequences may be homologous (homologs) if they share at least 20%, 25%, 30%. 35%, 40%, 45% 50%, 55%, 60%, 65%, 70%, 75%, 80%, 85%, 90%, 91%, 92%, 93%, 94%, 95%, 96%, 97%, 98%, 99%, or higher identity to a reference sequence when compared and aligned for maximum correspondence over a comparison window, or designated region as measured using one of the following sequence comparison algorithms or by manual alignment and visual inspection. In some cases, 2 or more sequences 2 or more sequences may be homologous if they share at most 20%, 25%, 30%. 35%, 40%, 45% 50%, 55%, 60%, 65%, 70%, 75%, 80%, 85%, 90%, 91%, 92%, 93%, 94%, 95%, 96%, 97%, 98%, 99% or higher identity to a reference sequence. This definition also refers to the compliment of a test sequence. Preferably, the identity exists over a region that is at least 25 amino acids or nucleotides in length or in some cases over a region that is 50-100 amino acids or nucleotides in length. In some cases, 2 or more sequences may be homologous and share at least 30% identity over at least 80 amino acids in a sequence according to the Sander-Schneider homology limit.

The term “incorporating” or “incorporated” as used herein with reference to a liposome means encapsulated/encapsulating into the cavity of the liposome, within the potential double layer of the liposome, or as part of the membrane layer of the liposome.

The term “inhibit” as used herein means to reduce by a measurable amount. The amount of reduction may vary and include, for example, a 1% to about a 99% reduction, a 1% to about a 95% reduction, a 1% to about a 90% reduction, a 1% to about a 85% reduction, a 1% to about a 80% reduction, a 1% to about a 75% reduction, a 1% reduction to about a 70% reduction, a 1% reduction to about a 65% reduction, a 1% reduction to about a 60% reduction, a 1% reduction to about a 55% reduction, a 1% reduction to about a 50% reduction, a 1% reduction to about a 45% reduction, a 1% reduction to about a 40% reduction, a 1% reduction to about a 35% reduction, a 1% reduction to about a 30% reduction, a 1% reduction to about a 25% reduction, a 1% reduction to about a 20% reduction, a 1% reduction to about a 15% reduction, a 1% reduction to about a 10% reduction, a 1% to about a 5% reduction, about a 5% to about a 99% reduction, about a 10% to about a 99% reduction, about a 15% to about a 99% reduction, about a 20% to about a 99% reduction, about a 25% to about a 99% reduction, about a 30% to about a 99% reduction, about a 35% to about a 99% reduction, about a 40% to about a 99% reduction, about a 45% to about a 99% reduction, about a 50% to about a 99% reduction, about a 55% to about a 99% reduction, about a 60% to about a 99% reduction, about a 65% to about a 99% reduction, about a 70% to about a 99% reduction, about a 75% to about a 95% reduction, about a 80% to about a 99% reduction, about a 90% reduction to about a 99% reduction, about a 95% to about a 99% reduction, about a 5% to about a 10% reduction, about a 5% to about a 25% reduction, about a 10% to about a 30% reduction, about a 20% to about a 40% reduction, about a 25% to about a 50% reduction, about a 35% to about a 55% reduction, about a 40% to about a 60% reduction, about a 50% reduction to about a 75% reduction, about a 60% reduction to about 80% reduction, or about a 65% to about a 85% reduction etc.), this is indicative of responsiveness

The term “immune response” as used herein refers to a response of a cell of the immune system, such as a B cell, T cell, dendritic cell, macrophage or polymorphonucleocyte, to a stimulus such as an antigen or vaccine. An immune response can include any cell of the body involved in a host defense response, including for example, an epithelial cell that secretes an interferon or a cytokine. An immune response includes, but is not limited to, an innate and/or adaptive immune response. As used herein, a protective immune response refers to an immune response that protects a subject from infection (e.g., prevents infection or prevents the development of disease associated with infection). Methods of measuring immune responses are well known in the art and include, for example, by measuring proliferation and/or activity of lymphocytes (such as B or T cells), secretion of cytokines or chemokines, inflammation, antibody production and the like. “Enhancing an immune response” refers to co-administration of an adjuvant and at least one peptide, wherein the adjuvant increases the desired immune response to the at least one peptide compared to administration of the at least one peptide in the absence of the adjuvant.

The term “immunogenic composition” as used herein are those which result in specific antibody production or in cellular immunity when injected into a subject. In certain embodiments, the vaccine disclosed elicits a neutralizing antibody response.

The term “immunogenic variants” as used herein refers to a variant that that is predicted to be immunogenic.

The term “increase” as used herein refers to an increase by a measurable amount. The amount of increase may vary and include, for example, a 1% to about a 99% increase, a 1% to about a 95% increase, a 1% to about a 90% increase, a 1% to about a 85% increase, a 1% to about a 80% increase, a 1% to about a 75% increase, a 1% increase to about a 70% increase, a 1% increase to about a 65% increase, a 1% increase to about a 60% increase, a 1% increase to about a 55% increase, a 1% increase to about a 50% increase, a 1% increase to about a 45% increase, a 1% increase to about a 40% increase, a 1% increase to about a 35% increase, a 1% increase to about a 30% increase, a 1% increase to about a 25% increase, a 1% increase to about a 20% increase, a 1% increase to about a 15% increase, a 1% increase to about a 10% increase, a 1% to about a 5% increase, about a 5% to about a 99% increase, about a 10% to about a 99% increase, about a 15% to about a 99% increase, about a 20% to about a 99% increase, about a 25% to about a 99% increase, about a 30% to about a 99% increase, about a 35% to about a 99% increase, about a 40% to about a 99% increase, about a 45% to about a 99% increase, about a 50% to about a 99% increase, about a 55% to about a 99% increase, about a 60% to about a 99% increase, about a 65% to about a 99% increase, about a 70% to about a 99% increase, about a 75% to about a 95% increase, about a 80% to about a 99% increase, about a 90% increase to about a 99% increase, about a 95% to about a 99% increase, about a 5% to about a 10% increase, about a 5% to about a 25% increase, about a 10% to about a 30% increase, about a 20% to about a 40% increase, about a 25% to about a 50% increase, about a 35% to about a 55% increase, about a 40% to about a 60% increase, about a 50% increase to about a 75% increase, about a 60% increase to about 80% increase, or about a 65% to about a 85% increase etc.), this is indicative of responsiveness.

The term “linker” as used herein refers to a molecule positioned between two moieties. Typically, linkers are bifunctional, i.e., the linker includes a functional group at each end, wherein the functional groups are used to couple the linker to the two moieties.

The term “lipid” as used herein refers to any suitable material resulting in a bilayer such that the hydrophobic portion of the lipid material orients toward the bilayer interior while the hydrophilic portion orients toward the aqueous phase. Hydrophilic characteristics derive from the presence of phosphato, carboxylic, sulfato, amino, sulfhydryl, nitro, and other like groups. Hydrophobicity could be conferred by the inclusion of groups that include, but are not limited to, long chain saturated and unsaturated aliphatic hydrocarbon groups and such groups substituted by one or more aromatic, cycloaliphatic or heterocyclic group(s).

The term “lipid bilayer” as used herein refers to any double layer of oriented amphipathic lipid molecules in which the hydrocarbon tails face inward to form a continuous non-polar phase.

The term “liposome” as used herein refers to a vesicle made of concentric bilayers of lipids and more particularly, non-phospholipids. The liposome can be formed of the same lipid or different lipids. These lipids can have an anionic, cationic or zwitterionic hydrophilic head group. The size of a liposome may vary but is generally from about 10 to about 3000 nm. In certain embodiments, the liposome has an aqueous core, while in other embodiments, the liposome has an oil-filled core. The term “empty liposome” as used herein refers to a liposome not incorporating any peptide or other antigen within the liposome core. In certain embodiments, the liposome is a non-phospholipid liposome.

The term “multimellar” as used here refers to a vesicle containing more than one lipid bilayers. In certain embodiments, the multimellar vesicle disclosed herein comprises two or more lipid bilayers, three or more lipid bilayers, four or more lipid bilayers, five or more lipid bilayers, six or more lipid bilayers, seven or more lipid bilayers, eight or more lipid bilayers, nine or more lipid bilayers, or ten or more lipid bilayers.

The term “multimeric” as used herein with reference to a peptide antigen refers to a structure consisting of several peptides that are associated covalently or non-covalently, with or without linkers. In certain embodiments, the multimeric peptide consists of at least two peptides (e.g., a dimer), at least three peptides (e.g., a timer). If all the subunits are the same, these are called homomeric peptides. Homomeric proteins consist of the same kind of subunits that are held together by noncovalent bonds to form a bigger, whole structure (i.e. quaternary structure). the subunits are different, these are called heteromeric proteins The term “non-ionic surfactant” as used herein refers to a class of surfactants which have no charge groups in their hydrophilic heads. In solutions, nonionic surfactants form structures in which hydrophilic heads are opposite to aqueous solutions and hydrophilic tails are opposite to organic solutions. Representative, non-limiting non-ionic surfactants include alkyl esters, alkyl amides, alkyl ethers and esters of fatty acids.

The term “paucimellar” as used herein refers to a vesicle having 2-10 lipid bilayers. In certain embodiments disclosed herein the vesicle that comprises the one or more peptides is paucimellar.

The term “peptide” as used herein refers to a sequence of two (2) or more amino acids and typically less than one hundred twenty (120) amino acids, wherein the amino acids are naturally occurring or non-naturally occurring amino acids. Non-naturally occurring amino acids refer to amino acids that do not naturally occur in vivo but which, nevertheless, can be incorporated into the peptide structures described herein. In some embodiments, a peptide can be between 2 and 10, about 8 and 40, about 12 and 60 or about 20 and about 80 amino acids in length. In certain embodiments, the peptide is 2, 3, 4, 5, 6, 7, 8, 9, 10, 11 or 12 amino acids in length. Various techniques are known for the preparation of peptides. For example, recombinant DNA technology or chemical synthesis can be used to prepare peptides disclosed herein. Peptides disclosed herein can be synthesized individually, or as longer polypeptides including two or more peptides. Peptides of disclosed herein can be isolated from host cells or synthesis reaction products after they are produced in the host cells using recombinant DNA technology or after they are chemically synthesized. That is, peptides disclosed herein can be purified or isolated so as not to substantially contain other host-cell proteins and fragments thereof, or any other chemical substance. In certain embodiments, herein, the peptide is a synthesized peptide.

The term “peptide antigen” as used herein refers to peptide that can stimulate the production of antibodies or a T cell response in an animal. A peptide antigen contains an epitope that can react with the products of specific humoral or cellular immunity to induce an immune response to the epitope. “Epitope” refers to the region of a peptide antigen to which B and/or T cells respond.

The term “pharmaceutical composition” refers to a mixture of one or more chemicals, or pharmaceutically acceptable salts thereof, with a suitable carrier, for administration to a mammal as a medicine.

The term “phospholipid” as used herein refers to any of a group of lipids whose molecule has a hydrophilic “head” containing a phosphate group, and two hydrophobic “tails” derived from fatty acids, joined by a glycerol molecule. The phosphate group can be modified with simple organic molecules such as choline, ethanolamine or serine. In certain embodiments herein, the liposome does not contain phospholipids.

The terms “polypeptide” and “protein” are terms that are used interchangeably to refer to a polymer of amino acids, without regard to the length of the polymer. Typically, polypeptides and proteins have a polymer length that is greater than that of “peptides.”

The term “prophylactic” as used herein with reference to an immunogenic composition (e.g., a vaccine) that is administered to a subject who does not exhibit signs of a disease.

The term “prophylactic vaccine” as used herein refers to a treatment that introduces an antigen into a patient with the goal that the patient's immune system will create antibodies for the antigen and increase or improve the subject's immune response to the associated illness or virus. In other words, a vaccinated subject will have a higher degree of resistance to illness or disease from the associated virus as compared to a non-vaccinated subject. This resistance may be evident by a decrease in severity or duration of symptoms of illness, decrease or elimination of viral shedding, and in some case the prevention of observable symptoms of infection in the vaccinated subject. In embodiments, a patient treated with a prophylactic vaccine does not have antibodies for the antigen prior to the treatment with the prophylactic vaccine (otherwise stated, the patient is “antibody naive”).

The term “protein” as used herein refers to a sequence of amino acid residues more than 120 amino acids in length.

The term “recombinant” as used herein refers to intended to refer to peptides, polypeptides or proteins that are designed, engineered, prepared, expressed, created, or isolated by recombinant means, such as polypeptides expressed using a recombinant expression vector transfected into a host cell, polypeptides isolated from a recombinant, combinatorial polypeptide library, or polypeptides prepared, expressed, created or isolated by any other means that involves splicing selected sequence elements to one another. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source. In some embodiments, one or more such selected sequence elements results from the combination of multiple (e.g., two or more) known sequence elements that are not naturally present in the same polypeptide.

The term “saturated” or “unsaturated”, when referring to a lipid or liposome, means that the lipid or lipid components of the liposome is a saturated or unsaturated compound. A saturated compound has only single bonds between carbon atoms and resists the addition reactions, such as hydrogenation, oxidative addition, and binding of a Lewis base. An unsaturated compound has at least one double bond. A saturated lipid in general has a higher melting temperature than comparable, unsaturated lipid. In some embodiments, saturated lipids increase entrapment stability of compounds (e.g., peptides).

The term “spike protein”, as used herein, refers to a type I transmembrane glycoprotein that is characteristic of coronaviruses. Most spike proteins contain a leader, an ectodomain, a transmembrane domain and an intracellular tail.

The term “subject in need thereof” as used herein refers to a living organism suffering from or prone to a disease or condition that can be treated by using the methods provided herein. The term does not necessarily indicate that the subject has been diagnosed with a particular disease or disorder, but typically refers to an individual under medical supervision. Non-limiting examples include humans, other mammals, bovines, rats, mice, dogs, monkeys, goat, sheep, cows, deer, and other non-mammalian animals. In embodiments, a patient is human.

The term “substitution” as used herein with reference to a peptide refers to the replacement of one amino acid residue by a different amino acid residue. In certain embodiments, the substitution is conservative. Conservative amino acid substitutions include: (i) small aliphatic, nonpolar or slightly polar residues: Ala, Ser, Thr, Pro, Gly; (ii) polar, negatively charged residues and their amides and esters: Asp, Asn, Glu, Gln, cysteic acid and homocysteic acid; (iii) polar, positively charged residues: His, Arg, Lys; Ornithine (Orn); (iv) large, aliphatic, nonpolar residues: Met, Leu, Ile, Val, Cys, Norleucine (Nle), homocysteine; and (iv) large, aromatic residues: Phe, Tyr, Trp, acetyl phenylalanine

The term “therapeutically effective amount” as used herein refers an amount sufficient to prevent, correct and/or normalize an abnormal physiological response. In one aspect, a “therapeutically effective amount” is an amount sufficient to reduce by at least about 30 percent, more preferably by at least 50 percent, most preferably by at least 90 percent, a clinically significant feature of pathology, such as for example, size of a tumor mass.

The term “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture.

The term “therapeutic vaccine” as used herein refers to a treatment that introduces an antigen into a patient that already has the associated illness or virus, with the goal that the patient's immune system will create antibodies for the antigen enabling the patient's body to fight harder against the illness or virus that it already has.

The terms “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made. Treatment includes preventing the disease, that is, causing the clinical symptoms of the disease not to develop by administration of a protective composition prior to the induction of the disease; suppressing the disease, that is, causing the clinical symptoms of the disease not to develop by administration of a protective composition after the inductive event but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance.

The term “vaccine” as used herein, refers to any type of biological preparation contributing to or soliciting active immune responses against a particular disease or pathogen. Such biological preparation can include, but is not limited to, an antigen derived from a disease-causing agent or a portion of an antigen derived from a disease-causing agent.

The term “vaccination” or “vaccinate” refers to the administration of a composition intended to generate an immune response, for example to a disease-causing agent. Vaccination can be administered before, during, and/or after exposure to a disease-causing agent, and/or to the development of one or more symptoms, and in some embodiments, before, during, and/or shortly after exposure to the agent. In some embodiments, vaccination includes multiple administrations, appropriately spaced in time, of a vaccinating composition.

The term “vaccine efficacy” or “VE” as used herein measure the proportionate reduction in cases among vaccinated persons. It is measured by calculating the risk of disease among vaccinated and unvaccinated persons and determining the percentage reduction in risk of disease among vaccinated persons relative to unvaccinated persons. The greater the percentage reduction of illness in the vaccinated group, the greater the vaccine efficacy.

The term “variant” as used refers to a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence. A “variant” of a protein or peptide may have at least 70%, 75%, 80%, 85%, 90%, 95%, 98% or 99% amino acid identity over a stretch of 10, 20, 30, 50, 75 or 100 amino acids of such protein or peptide. Variants of the proteins or peptides disclosed herein, which may be encoded by a nucleic acid molecule, may also comprise those sequences, wherein nucleotides of the encoding nucleic acid sequence are exchanged according to the degeneration of the genetic code, without leading to an alteration of the respective amino acid sequence of the protein or peptide, i.e. the amino acid sequence or at least part thereof may not differ from the original sequence in one or more mutation(s) within the above meaning.

Representative, non-limiting variants of SARS-COV-2 include alpha (B.1.1.7 and Q lineages), beta (B.1.351 and descendent lineages), delta (B.1.617.2 and AY lineages) gamma (P.1 and descendent lineages), epsilon (B.1.427 and B.1.429), Eta (B.1.525, iota (B.1.526), kappa (B.1.617.1), 1.617.3, omicron (B.1.1.529 and BA lineages), mu (B.1.621, B.1.621.1) and zeta (P.2). In certain embodiments, the compositions (e.g., vaccines) discloses herein contain one more peptides from SARS-COV-2, variants of SARS-COV-2 or combinations thereof.

The term “vesicle” as used herein refers to a structure consisting of liquid or cytoplasm enclosed by a lipid bilayer. The interior of the vesicle is typically an aqueous environment but can also be an oily environment, and it may comprise an agent such as but not limited to a prophylactic, therapeutic or diagnostic agent.

### II. Immunogenic Compositions

The present disclosure provides immunogenic compositions (e.g., vaccines) and pharmaceutical compositions comprising the comprise at least one peptide (e.g., a synthetic peptide) and a liposome (e.g., a non-phospholipid liposome incorporating Vitamin E). In certain embodiments, the at least one peptide is encapsulated within the liposome. These compositions are suitable for use, for example, in generating an immune response as described further herein.

A. Synthetic Peptide

The immunogenic compositions (e.g., vaccines) disclosed herein include at least one peptide, such as a synthetic immunogenic peptide. In certain embodiment, the immunogenic compositions contain a combination of peptides. In certain embodiments, the immunogenic composition cross-reacts with the modified RBD and modified S1 protein of SARS-CoV-2.

In one embodiment, the at least one peptide is a linear peptide. In certain embodiments, the immunogenic composition comprises at least two synthetic immunogenic linear peptides, where the at least two synthetic immunogenic linear peptides can be the same or different (i.e., mixed).

In one embodiment, the peptide is a multimeric peptide. In certain embodiments, the multimeric peptide has a linear, branched, dendrimer or star-like structure. The multimeric peptide may be homomeric or heteromeric.

In certain embodiments, the immunogenic composition comprises at least three, at least four, at least five, at least six, at least seven, at least eight, at least nine or at least two or more peptides. The one or more peptides may be the same or different.

In certain embodiments, the immunogenic composition comprises two or more peptides that differ by sequence. The ratio of the different peptides may be, for example, 1:1, 2:1, 3:1 or 4:1.

In a particular embodiment, the immunogenic composition comprises at least one (e.g., one, two, three, four or more) peptides derived from viral proteins, and more particularly viral proteins of SARS-CoV-2 or HIV.

In a particular embodiment, the immunogenic composition comprises at least one (e.g., one, two, three, four or more) peptides derived from the spike (S) protein of SARS-CoV-2 and more particularly, the Receptor Binding Motif (RBM) of the S protein. The at least one peptide may be a peptide that is currently known or unknown, i.e., occurs currently or represents a predicted mutation.

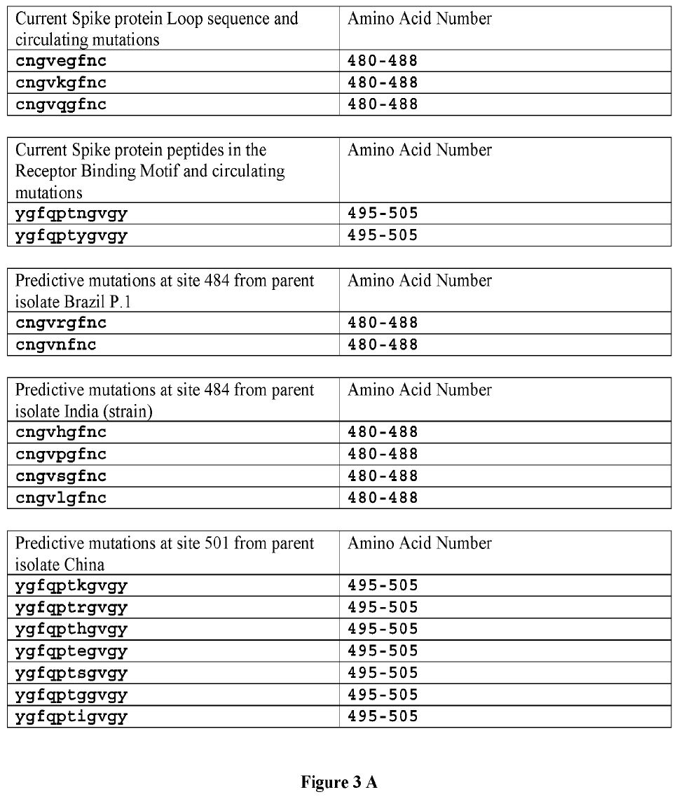

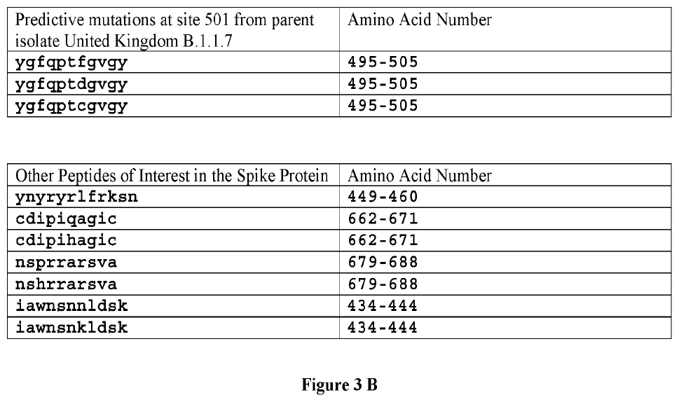

In one embodiment, the immunogenic composition comprises at least one peptide shown in FIGS. 3A and/or 3B.

In one embodiment, the immunogenic composition comprises two or more linear peptides derived from SEQ ID. NO: 1 or a variant or homolog thereof, for example, a variant or homolog comprising one or more substitutions.

In one embodiment, the immunogenic composition comprises a multimeric peptide comprising two or more peptides derived from SEQ ID. NO: 1 or a variant or homolog thereof, for example, a variant or homolog comprising one or more substitution mutations.

In certain embodiments, the immunogenic composition comprises a multimeric peptide comprising two or more peptides comprising amino acids 480-488 and 490-495-505 of SEQ ID NO: 1, or variant or homolog thereof, for example, a variant or homolog comprising one or more substitution mutations.

In a particular embodiment, the immunogenic compositions comprises of two or more linear peptides selected from the group consisting of cngvegfnc (SEQ ID NO: 6), ygfqptngvgy (SEQ ID NO: 7), cngvKgfnc (SEQ ID NO: 8), ygfqptYgvgy (SEQ ID NO: 9) and combinations thereof. In certain embodiments, the two or more peptides are variants or homologs of such sequences.

In a particular embodiment, the immunogenic composition comprises a multimeric peptide comprising of two or more monomers selected from the group consisting of cngvegfnc (SEQ ID NO: 6), ygfqptngvgy (SEQ ID NO: 7), cngvKgfnc (SEQ ID NO: 8), ygfqptYgvgy (SEQ ID NO: 9) and combinations thereof. In certain embodiments, the two or more peptides or variants or homologs of such sequences.

In certain embodiments, the multimeric peptide is a septamer comprising the following peptides: cngvegfnc (SEQ ID NO: 6), ygfqptngvgy (SEQ ID NO: 7), cngvKgfnc (SEQ ID NO: 8), ygfqptYgvgy (SEQ ID NO: 9), including in combination. In certain embodiments, the two or more peptides are variants or homologs of such sequences.

The multiple peptides comprising the multimeric peptide embodiment described herein may be attached covalently or non-covalently, by linkers or without linkers.

Representative, non-limiting linkers include simple covalent bond, a flexible peptide linker, a disulfide bridge or a polymer such as polyethylene glycol (PEG). Peptide linkers may be entirely artificial (e.g., comprising 2 to 20 amino acid residues independently selected from the group consisting of glycine, serine, asparagine, threonine and alanine) or adopted from naturally occurring proteins. Disulfide bridge formation can be achieved, e.g., by addition of cysteine residues, as further described herein below. Linking through polyethylene glycols (PEG) can be achieved by reaction of monomers having free cysteines with multifunctional PEGs, such as linear bis-maleimide PEGs. Alternatively, linking can be performed though the glycans on the monomer after their oxidation to aldehyde form and using multifunctional PEGs containing aldehyde-reactive groups.

The peptides disclosed herein may optionally comprise non-amino acid moieties, e.g., hydrophobic moieties (various linear, branched, cyclic, polycyclic or heterocyclic hydrocarbons and hydrocarbon derivatives) attached to the peptides as well as various protecting groups. Chemical (non-amino acid) groups may be included in order to improve certain physiological properties such; decreased degradation or clearance; decreased repulsion by various cellular pumps, improve immunogenic activities, improve various modes of administration (such as attachment of various sequences which allow penetration through various bathers, through the gut, etc.); increased specificity, increased affinity, decreased toxicity and the like.

In certain embodiments, the at least one synthetic peptide is a multimeric peptide having or more improved properties compared to a monomer peptide, including enhanced antigen immunogenicity, binding affinity, cytotoxic potency, and/or selectivity.

In a particular embodiment, antigen immunogenicity of the multimeric peptide has at least about 5%, at least about 10%, at least about 15%, at least about 20%, at least about 25%, at least about 30%, at least about 35%, at least about 40%, at least about 45% or at least about 50% or more enhanced immunogenicity compared to the monomer peptide(s).

In a particular embodiment, antigen immunogenicity of the multimeric peptide has at least about 5%, at least about 10%, at least about 15%, at least about 20%, at least about 25%, at least about 30%, at least about 35%, at least about 40%, at least about 45% or at least about 50% or more binding affinity compared to the monomer peptide(s).

In a particular embodiment, antigen immunogenicity of the multimeric peptide has at least about 5%, at least about 10%, at least about 15%, at least about 20%, at least about 25%, at least about 30%, at least about 35%, at least about 40%, at least about 45% or at least about 50% or more selectivity compared to the monomer peptide(s).

In certain embodiments, the adjuvanted peptide vaccines disclosed herein have one or more improved properties compared to a monomer peptide, including enhanced antigen immunogenicity, binding affinity, cytotoxic potency, and/or selectivity compared to a non-adjuvanted version of the same peptide vaccine.

In a particular embodiment, the adjuvanted peptide vaccine has at least about 5%, at least about 10%, at least about 15%, at least about 20%, at least about 25%, at least about 30%, at least about 35%, at least about 40%, at least about 45% or at least about 50% or more enhanced immunogenicity compared to a non-adjuvanted version of the same peptide vaccine.

In a particular embodiment, the adjuvanted peptide vaccine has at least about 5%, at least about 10%, at least about 15%, at least about 20%, at least about 25%, at least about 30%, at least about 35%, at least about 40%, at least about 45% or at least about 50% or more binding affinity compared to a non-adjuvanted version of the same peptide vaccine.

In a particular embodiment, the adjuvanted peptide vaccine has at least about 5%, at least about 10%, at least about 15%, at least about 20%, at least about 25%, at least about 30%, at least about 35%, at least about 40%, at least about 45% or at least about 50% or more selectivity compared to a non-adjuvanted version of the same peptide vaccine.

The synthetic peptide may be any suitable synthetic peptide or antigen. The synthetic peptide may be derived, for example, by an infectious agent selected from a virus, bacteria or fungi.

In one embodiment, the at least one synthetic peptide is derived from viral peptide or antigen. Virion particles are generally comprised of genetic material (e.g., DNA or RNA), a protein coat, and a lipid envelope and use receptors and co-receptors to enter a cell.

In a particular embodiment, the at least one synthetic peptide is derived from a viral peptide from a double stranded DNA virus (dsDNA), a single stranded DNA virus (ssDNA), a double stranded RNA virus (dsRNA) or a single stranded RNA virus (ssRNA).

In one embodiment, the viral peptide is derived from a DNA virus selected from the group consisting of adenovirus, papillomavirus, parvovirus, herpes simplex virus, varicella-zoster virus, cytomegalovirus, Epstein-Barr virus, smallpox virus, vaccinia virus, and hepatitis B virus.

In another embodiment the viral peptide derived from RNA virus selected from the group consisting of tavirus, norovirus, enterovirus, hepatovirus, rubella virus, influenza viruses (A, B, and C), measles virus, mumps virus, hepatitis C virus, yellow fever virus, hantavirus, Zika virus, California encephalitis virus, rabies virus, ebola virus, and HIV.

### ii. Coronavirus

In one embodiment, the viral peptide is from a coronavirus. The coronavirus can be any coronavirus currently known, or later discovered. In certain embodiments, the coronavirus is zoogenic.

Coronaviruses are positive strand RNA viruses with the largest viral genome among the RNA viruses (27-33 kb). The virus particles are enveloped and carry extended spike proteins on the membrane surface, providing the typical crown-like structure. The coronaviruses share a conserved organization of their (positive strand) RNA genome. The 5′ two-thirds of the genome contains the large 1a and 1b ORFs, encoding the proteins necessary for RNA replication (the nonstructural proteins), whereas the 3′ one-third contains the genes coding for the structural proteins: haemagglutinin esterase protein (only for group IIa), spike protein, envelope protein, membrane protein and nucleocapsid protein. The accessory protein genes are interspersed between the structural protein genes and differ in number and position for the various coronavirus.

Several coronavirus genera and subgenera are recognized (https://talk.ictvonline.org/ictv-reports/). Among them, alpha- and betacoronaviruses infect mammals, gammacoronaviruses infect avian species, and deltacoronaviruses infect both mammalian and avian species. The coronavirus may be, for example, 229E, SARS, MERS, SARS-CoV-1 (OC43), and SARS-CoV-2.

The coronavirus spike protein includes three segments: a large ectodomain, a single-pass transmembrane anchor, and a short intracellular tail The ectodomain consists of a receptor-binding subunit S1 and a membrane-fusion subunit S2. The S1 and S2 domains may be separated by a cleavage site that is recognized by furin-like proteases during S protein biogenesis in the infected cell. The spike protein binds to a receptor on the host cell surface through the S1 subunit and then fuses viral and host membranes through its S2 subunit. The spike protein exists in two structurally distinct conformations, pre-fusion and post-fusion.

The S1 subunit of the betacoronavirus spike proteins displays a multidomain architecture and is structurally organized in four distinct domains A-D of which domains A and B may serve as a Receptor Binding Domain (RBD).

In a particular embodiment, the viral peptide is from SARS-CoV-2 or a variant thereof. SARS-CoV-2 can cause severe respiratory illness and significant mortality among those over 60 years of age or with chronic conditions. Infection of target cells by SARS CoV-2 is mediated through the interaction of the viral Spike (S) protein (1255 amino acids) and its cellular receptor, angiotensin-converting enzyme 2 (ACE2). The SARS CoV receptor-binding domain (amino acids N318-T509) includes a region along its periphery that contacts ACE2 and is designated the receptor-binding motif (RBM, amino acids 5432-T486).

In one embodiment, the viral peptide is derived from the spike (S), envelope (E), membrane (M), or nucleocapsid (N) protein of the coronavirus or a combination thereof.

In one embodiment, the vaccine contains at least one synthetic peptide derived from the spike protein and more particularly, the S1 domain (amino acid number 16-635 of the spike protein). In one embodiment, the at least one synthetic peptide is derived from a fragment of the S1 domain. The fragment of the S1 domain may include 1, 5, 10, 15, 20, 25 or 30 amino acids of the S1 domain. In a particular embodiment, the fragment includes less than 30 amino acids of the S1 domain, and more particularly, less than about 25, less than about 20, less than about 15 or less than about 10 amino acids of the S1 domain.

In certain embodiments, the vaccine contains at least one synthetic peptide derived from the Receptor Binding Domain (RBD) (amino acid number 319-541) of the spike protein.

In certain embodiments, the vaccine contains at least one peptide (e.g., one, two, three, four or more) derived from the RBD of SEQ. ID NO: 1, which is the SARS-CoV-2 Spike Protein sequence from the Shanghai, China Human Isolate collected on 2-18-2020 (Accession #QJG65958) from the National Center for Biotechnology Information protein database.

In one embodiment, the vaccine contains at least one peptide (e.g., one, two, three, four or more) derived from the RBD of SEQ. ID. NO: 1 at amino acids 480-488 cngvegfnc (Cysteine-Asparagine-Glycine-Valine-Glutamic Acid-Glycine-Phenylalanine-Asparagine-Cysteine) (SEQ ID NO: 6) and 495-505 ygfqptngvgy (Tyrosine-Glycine-Phenylalanine-Glutamine-Proline-Threonine-Asparagine-Glycine-Valine-Glycine-Tyrosine) (SEQ ID NO: 7).

There are multiple other mutations or deletions in the Spike protein, and other SARS-CoV-2 proteins, which would not be covered by current vaccines or prior immunity. Those mutations and deletions are identified in the Spike protein by red entries in the Spike protein sequences. Double mutation isolates have independently developed in three different continents in less than six months: Europe, Africa, and South America. These isolates are currently spreading to multiple countries to include the United States (https://www.cdc.gov/coronavirus/2019-ncov/transmission/variant-cases.html). Adjuvanted vaccines directed against these and other mutations which have occurred in less than one year from the initial infections in China will continue to occur both in the RBM and in other parts of the Spike protein, and other proteins in SARS-CoV-2.

In another embodiment, the two peptides are derived from SEQ ID NO:1 having one or more mutations, e.g., a substitution, insertion, deletion or inversion. In a particular embodiment, the variant sequence has at least 80%, 85%, 90%, 93%, 95%, 96%, 97%, 98%, 99%, 99.5%, 99.6%, 99.7%, 99.8%, 99.9%, 99.95%, 99.98% or 99.99% sequence identity to SEQ ID NO: 1.

In certain embodiments, the vaccine contains at least one peptide (e.g., one, two, three, four or more) derived from the RBD of the Wuhan isolate.

In certain embodiments, the vaccine contains at least one peptide (e.g., one, two, three, four or more) derived from the RBD of the South African isolate (B.1.351). SEQ. ID. NO.: 4.

In certain embodiments, the vaccine contains at least one peptide (e.g., one, two, three, four or more) derived from the RBD of the Brazilian isolate (B.1.351). SEQ. ID. NO.: 5.

In certain embodiments, the vaccine contains at least one peptide (e.g., one, two, three, four or more) derived from the RBD of the Cal-20C isolate.

In a particular embodiment, the vaccine contains at least one peptide from the United Kingdom variant (B.1.1.7). SEQ. ID. NO.: 2; SEQ. ID. NO.: 3.

In a particular embodiment, the vaccine contains at least one peptide from the gamma or (P.1), and omicron (B.1.1.529) variants of SARS-COV-2.

In a particular embodiment, the peptide is selected from the group consisting of cngvegfnc (SEQ ID NO: 6) (Peptide A), ygfqptngvgy (SEQ ID NO: 7) (Peptide B), cngvKgfnc (SEQ ID NO: 8), ygfqptYgvgy (SEQ ID NO: 9) or combinations thereof.

In another particular embodiment, the peptide is a multimeric peptide comprising seven peptide monomers selected from the group consisting of cngvegfnc (SEQ ID NO: 6), ygfqptngvgy (SEQ ID NO: 7), cngvKgfnc (SEQ ID NO: 8), ygfqptYgvgy (SEQ ID NO: 9) and combinations thereof.

In a particular embodiment, the peptide is a multimeric peptide comprising four copies of cngvegfnc (SEQ ID NO: 6), and three copies of ygfqptngvgy (SEQ ID NO: 7) connected by lysine linkers.

In some embodiments, the adjuvated peptide immunogenic composition or vaccine cross-reacts with the Receptor Binding Domain, and the S1 protein of SARS-CoV-2.

In certain embodiments, the at least one peptide is selected from the group consisting of current spike protein loop sequence (amino acids 480-488) and circulating mutations, e.g., cngvegfnc (SEQ ID NO: 6), cngvkgfnc (SEQ ID NO: 8), cngvqgfnc (SEQ ID NO: 10), and cngvqgfnc (SEQ ID NO: 10).

In certain embodiments, the at least one peptide is selected from the group consisting of current spike protein peptides in the Receptor Binding Motif (amino acids 495-505) and circulating mutations, e.g., ygfqptngvgy (SEQ ID NO: 7) and ygfqptygvgy (SEQ ID NO: 9).

In certain embodiments, the at least on peptide is selected from the group consisting of predictive mutations at site 484 (amino acids 480-488) from parent isolate Brazil P.1, e.g., cngvrgfnc (SEQ ID NO: 11) and cngvnfnc (SEQ ID NO: 12).

In certain embodiments, the at least on peptide is selected from the group consisting of predictive mutations at site 484 from parent isolate India (strain), e.g., cngvhgfnc (SEQ ID NO: 13), cngvpgfnc (SEQ ID NO: 14), cngvsgfnc (SEQ ID NO: 15), and cngvlgfnc (SEQ ID NO: 16).

In certain embodiments, the at least on peptide is selected from the group consisting of predictive mutations at site 501 from parent isolate China (amino acids 495-505), e.g., ygfqptkgvgy (SEQ ID NO: 17), ygfqptrgvgy (SEQ ID NO: 18), ygfqpthgvgy (SEQ ID NO: 19), ygfqptegvgy (SEQ ID NO: 20), ygfqptsgvgy (SEQ ID NO: 21), ygfqptggvgy (SEQ ID NO: 22) and ygfqptigvgy (SEQ ID NO: 23)

In certain embodiments, the at least one peptide is selected from the group consisting of predictive mutations at site 501 from parent isolate United Kingdom (B.1.1.7) (amino acids numbers 495-505) including ygfqptfgvgy (SEQ ID NO: 24), ygfqptdgvgy (SEQ ID NO: 25) and ygfqptcgvgy (SEQ ID NO: 26).

In certain embodiments, the at least one peptide is selected from other peptides of interest in the spike protein including ynyryrlfrksn (SEQ ID NO: 27) (amino acids 449-460), cdipiqagic (SEQ ID NO: 28) (amino acids 662-671), cdipihagic (SEQ ID NO: 29) (amino acids 662-671), nsprrarsva (SEQ ID NO: 30) (amino acids 679-688), nshrrarsva (SEQ ID NO: 31) (amino acids 679-688), iawnsnnldsk (SEQ ID NO: 32) (amino acids 434-444) and iawnsnkldsk (SEQ ID NO: 33) (amino acids) 434-444.

### iii. Retrovirus

In another embodiment, the at least one peptide is from a retrovirus.

Retroviruses a class of viruses of vertebrate animals in which the genetic material is RNA, instead of DNA. Such viruses are accompanied by a polymerase enzyme known as “reverse transcriptase,” which catalyzes transcription of viral RNA into DNA that is integrated into a host cell's genome. The resultant DNA may remain in a dormant state in an infected cell for an indeterminate period of time or become incorporated into the cell genome and actively cause the formation of new virions. The retrovirus may be an oncovirus, lentivirus or a spumarvirus.

In one embodiment, the at least one synthetic peptide is from HIV. In certain embodiments, the synthetic peptide is gp120 or gp41 or a fragment thereof.

Current therapy (highly active antiretroviral therapy or HAART) for controlling HIV-1 infection and impeding AIDS development profoundly reduces viral replication in cells that support HIV-1 infection and reduces plasma viremia to a minimal level. But HAART fails to suppress low level viral genome expression and replication in tissues and fails to target the latently-infected cells, for example, resting memory T cells, brain macrophages, microglia, and astrocytes, gut-associated lymphoid cells, that serve as a reservoir for HIV-1. Persistent HIV-1 infection is also linked to co-morbidities including heart and renal diseases, osteopenia, and neurological disorders.

B. Lipid Vesicle

In some embodiments, the at least one synthetic peptide is provided? with (e.g., encapsulated within) a lipid vesicle.

Lipid vesicles are substantially spherical structures made of amphiphiles, e.g., surfactants or phospholipids. The lipids of these spherical vesicles are generally organized in the form of lipid bilayers, e.g., multiple onion-like shells of lipid bilayers which encompass an aqueous volume between the bilayers. Certain types of lipid vesicles have an unstructured central cavity which can be used to encapsulate and transport a variety of materials. The lipid vesicle may be charged or neutral.

The lipid vesicle may be any suitable lipid vesicle such as a liposome, e.g., a non-phospholipid based liposome comprising an adjuvanting oil. The lipid vesicle may be a unimellar or multimellar vesicle. Multilamellar vesicles are concentric circles constructed by at least 2 bilayer vesicles or a large vesicle embodying one or more small vesicles.

In one embodiment, the lipid vesicle is a liposome. According to this embodiment, the liposome is formed of one or more phospholipids selected from the group consisting of dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“WPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DWG”), dipalmitoylphosphatidylglycerol (“DPPG”), di stearoylphosphatidylglycerol (“DSPG”), di stearoyl sphingomyelin (“DS SP”), di stearoylphophatidylethanolamine (“D SPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidyletlianolamine (“POPE”), lysophosphatidylcholine, lysophosphatidylethanolamine, and dilinoleoylphosphatidylcholine. The phospholipids may be synthetic or natural.

Liposome properties differ and may be selected on the basis of lipid composition, surface charge, size, and the method of preparation. Typically, liposomes can be divided into three categories based on their overall size and the nature of the lamellar structure. In one embodiment, the liposome is a small unilamellar vesicle (SUV) between about 20 and about 100 nm, a large unilamellar vesicle (LUV) greater than about 100 nm, a giant unilamelar vesicle (GULV) greater than about 100 nm, an oligomellar vesicle (OLV) between about 100 and about 1000 nm or a multilamellar large vesicle (MLV) greater than about 500 nm.

In certain embodiments, the lipid vesicle contains relatively few phospholipids, i.e., phospholipids are in the minority or absent compared to lipids as a whole.

In a particular embodiment, the lipid vesicle is a non-phospholipid vesicle comprising monoalkylated amphiphiles and a sterol. The monoaklyated amiphile may be, for example, •alpha-hydroxypalmitic acid, alpha-fluoropalmitic acid, cetylpyridium chloride, cetyltrimethylammonium bromide, diglyceryl monolaurate, lysoPC, myristic acid, N-myristoylethanolamine, N-palmitoylethanolamine, N-stearoylethanolamine, octadecyl methyl sulfoxide, palmitic acid (PA), polyoxyethylene alkylethers, stearic acid, stearylamine, tetraglyceryl monolaurate, Tween 20, Tween 21 or Tween 60. The sterol may be, for example, cholesterol, cholesterol sulfate, dihydrocholesterol, and 7-dehydrocholesterol stigmastanol, stigmasterol or ergosterol.

In certain embodiments, the sterol component is greater than about 60%, about 65%, about 70% or about 75% of the lipid vesicle.

The non-ionic surfactant may be any suitable surfactant include, e.g., a Span, a Tween or a Brij.

In certain embodiments, the one or more non-ionic surfactants are selected from of polyoxyethylene fatty acid esters, polyoxyethylene fatty acid ethers, polyoxyethylene sorbitan esters, polyoxyethylene glyceryl mono- and diesters, glyceryl mono- and distearate, sucrose distearate, propylene glycol stearate, long chain acyl hexosamides, long chain acyl amino acid amides, long chain acyl amides, glyceryl mono- and diesters, dimethyl acyl amines, C 12-C 20 fatty alcohols, C 12-C 20 glycol monoesters, and C 12-C 20 fatty acids.

In certain embodiments, the liposome further comprises polyoxyethylene fatty acid ethers (polyoxyethylene 2-stearyl or cetyl ethers), at least one sterol consisting of cholesterol as a membrane stabilizer, a negative charge producing agent (oleic acid), Vitamin E and any lipid soluble or water soluble materials to be incorporated into the vesicles.

In some embodiments, the liposome may also comprise squalene. In one embodiment, the squalene is non-mammalian squalene, e.g., plant or microorganism-derived squalene.

Liposome properties may differ and can be selected on the on the basis of lipid composition, surface charge, size, and the method of preparation.

In one embodiment, the lipid vesicle is a liposome selected from a small unilamellar vesicle (SUV) (10-100 nm), a large unilamellar vesicle (LUV) (100-3000 nm) and multi-lamellar vesicle (MLV). In certain embodiments, the liposome comprises between 2 and about 10 layers. The 2 to 10 bilayers encapsulate an aqueous volume which is interspersed between the lipid bilayers and may also be encapsulated in the amorphous central cavity. Alternatively, the amorphous central cavity may be substantially filled with a water immiscible material, such as an oil or wax. The paucilamellar vesicles containing such amphiphiles provide a high carrying capacity for water-soluble and water immiscible substances. The high capacity for water immiscible substances represents a unique advantage over classical phospholipid multilamellar liposomes.

The lipid vesicle contains a central cavity, carrying either water soluble materials or a water-immiscible oily solution, which can be used to encapsulate the antigen. The water-immiscible oily solution is made of materials which are both water immiscible and immiscible in the lipids used to form the bilayers. The water immiscible oily material found in the amorphous central cavity may comprise of Vitamin E or squalene oil.

In certain embodiments, oleic acid can insert in the membrane allowing negatively charged structures to be produced.

C. Pharmaceutical Composition

In certain embodiments, the immunogenic composition (e.g., vaccine) includes a pharmaceutically acceptable carrier. The pharmaceutically acceptable carrier may be formulated for administration to a human subject or patient.

In some embodiments, the pharmaceutically acceptable carrier includes solvents, dispersion media, coatings, stabilizing agents, diluents, preservatives, antibacterial and antifungal agents, isotonic agents, adsorption delaying agents, adjuvants, immune stimulants, and combinations thereof. Dilutents include, for example, water, saline, dextrose, ethanol, glycerol, and the like. Isotonic agents can include sodium chloride, dextrose, mannitol, sorbitol, and lactose. Stabilizers include, for example, albumin and alkali salts of ethylendiamintetracetic acid.

Compositions may be prepared as injectables, either as liquid solutions or suspensions. Solid forms suitable for solution in, or suspension in, liquid vehicles prior to injection can also be prepared (e.g. a lyophilized composition or a spray-freeze dried composition). The composition may be prepared for topical administration e.g. as an ointment, cream or powder. The composition may be prepared for oral administration e.g. as a tablet or capsule, as a spray, or as a syrup (optionally flavored). The composition may be prepared for pulmonary administration e.g. as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a mammal Such kits may comprise one or more antigens in liquid form and one or more lyophilized antigens. Compositions may be presented in vials, or they may be presented in ready-filled syringes. The syringes may be supplied with or without needles. A syringe will include a single dose of the composition, whereas a vial may include a single dose or multiple doses.

The composition may be packaged in unit dose form or in multiple dose form. For multiple dose forms, vials are preferred to pre-filled syringes.

### III. Methods of Use

The immunogenic compositions (e.g., vaccines) described herein are useful, for example, for generating an immune response. One method includes contacting a cell with an effective amount of the immunogenic composition described herein.

In some embodiments, methods of inducing an immune response are used for vaccination. The methods involve administering a therapeutically or prophylactically-effective amount of the immunogenic composition as described herein to treat, cure or prevent an infection by, or an amount sufficient to reduce the biological activity of, of an infectious agent such as a virus (e.g., a coronavirus or retrovirus).

In certain embodiments, the vaccine is a prophylactic vaccine, i.e., confers immunity to a subject who is not infected. According to this embodiment, the method comprises administering the vaccine to a subject in need thereof. In certain embodiments, administration is subcutaneously or intramuscularly.

For example, and without limitation, the one or more subsequent exposures occurring following/after administration may result in reduced viral titers, reduced amount and/or severity of symptoms, shortened duration of symptoms, and/or reduced need for treatment medications and/or clinician oversight, as compared to a control. In certain embodiments, the vaccine efficacy is about 60% or more, about 65% or more, about 70% or more, about 80% or more, about 85% or more, about 90% or more, or about 90% or more.

In a particular embodiment, the vaccine efficacy is about 91%, about 92%, about 93%, about 94%, about 95%, about 96%, about 97%, about 98%, about 99% or about 100%.

In certain embodiments, the vaccine is a therapeutic vaccine, i.e., is provided to a subject who has been diagnosed with an infection such as a viral infection. According to this embodiment, the method comprising administering the vaccine to a subject in need thereof. In certain embodiments, administration is subcutaneous or intramuscular.

In certain embodiments, administration of the vaccine to a subject in need thereof reduces the infection by at least 25%, at least 50%, or at least 75% as compared to a control.

In one embodiment, administration of the vaccine to a subject in need thereof results in a reduction in viral load of at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30% at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, at least about 98%, at least about 99%, or by 100%.

In one embodiment, administration of the vaccine to a subject in need there of results in a reduction in one or more symptoms or clinical signs of infection. These signs may include, for example, cough, fever, shortness of breath, fatigue, muscle aches or headaches. In certain embodiments, reduction in symptoms or signs is, compared to a control, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30% at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, at least about 98%, at least about 99%, or by 100%.

In one embodiment, administration of the therapeutic vaccine to a subject in need thereof reduces hospital stays by about 1, about 2, about 3, about 4, about 5, about 6 or about 7 days or more compared to control.

In one embodiment, administration of the therapeutic vaccine to a subject in need thereof reduces mortality compared to a control.

In certain embodiment, administration of the immunogenic composition (e.g., vaccine) as reduced side effects in comparison to other known immunogenic compositions (e.g., vaccines), such as those directed to the same infectious agent.

In a particular embodiment, one or more side effects are reduced selected from blood clots, allergic reaction (e.g., severe allergic reaction), anaphylaxis, Guillain-Barre syndrome, myocarditis, pericarditis or combinations thereof.

In certain embodiments, the immunogenic composition (e.g., vaccine) is administered to a subject as a single dose followed by a second dose later and optionally even a third, fourth (or more) dose subsequent thereto etc. A booster dose may also be administered. In one embodiment the immunogenic composition (e.g., vaccine) and/or booster administrations may be repeated and such administrations may be separated by at least 1 day, 2 days, 3 days, 4 days, 5 days, 10 days, 15 days, 30 days, 45 days, 2 months, 75 days, e.g. 1 to 5 days, 1 to 10 days, 5 to 15 days, 10 to 20 days, 15 to 25 days, 20 to 30 days, 25 to 35 days, 30 to 50 days, 40 to 60 days, 50 to 70 days, 1 to 75 days, or 1 month, 2 months, 3 months, 4 months, 5 months, or at least 6, 7, 8, 9, 10, 11, 12 months, 18 months, 24 months, 30 months, 36 months, 1 year, 2 years, 3 years, 5 years, 10 years, 15 years, 20 years, 30 years, 40 years, 50 years, 60 years, or even more. In certain aspects, the inventive vaccine may be administered to a subject as a single dose once per year.

In one embodiment, the vaccine disclosed herein is administered as a booster to one or more vaccines known in the art. In a particular embodiment, the vaccine disclosed herein is administered as a booster to an mRNA vaccine, a protein subunit vaccine, a non-replicating viral vector vaccine or an inactivated vaccine. In a particular embodiment, the vaccine disclosed herein is administered as a booster to a vaccine specific to a particular strain of a virus or, alternatively, a multi-strain vaccine. In a particular embodiment, the vaccine disclosed herein is a booster to current first-generation SARS-CoV-2 vaccines selected from the Pfizer-BioNTech COVID-19 vaccine (Comirnaty), the Moderna COVID-19 vaccine (Spikevax), the Johnson & Johnson vaccine (Ad26.COV2.S), or other COVID-19 vaccines.

In a particular embodiment, the vaccine disclosed herein is a booster for a vaccine selected from the group consisting of the Novavax vaccine (Nuvaxovid), the Serum Institute of India vaccine (COVOVAX, Covishield), the Oxford AstraZeneca vaccine (Vaxzevria), the Bharat Biotech vaccine (Covaxin), the Sinopharma vaccine (Covilo) or the Sinovac vaccine (Coronavac).

In certain embodiments, the vaccine is administered therapeutically in combination with at least one therapeutic agent. As used herein, the term “in combination,” in the context of the administration of two or more therapies to an elderly patient as defined herein, refers to the use of more than one therapy, preferably two therapies or even more. The use of the term “in combination” does not restrict the order in which therapies are administered to an elderly patient as defined herein. For example, a first therapy (e.g., a first prophylactic or therapeutic agent) can be administered at any time prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to an elderly patient as defined herein.

Non-limiting examples of therapeutic agents that may be administered in combination with the immunogenic composition (e.g., vaccine) disclosed herein include antibodies, aptamers, adjuvants, anti-inflammatories, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

In a particular embodiment, the therapeutic agent administered in combination with the immunogenic composition is selected from remdesivir (Veklury), nirmatrelvir and ritonavir (Paxlovid) and molnupiravir (Lagevrio).

In certain embodiments, the immunogenic composition disclosed herein is administered in combination with a monoclonal antibody treatment for example casirivimab and imdevimab (REGEN-COV), sotrovimab (or bamlanivimab and etesevimab.

In a particular embodiment, the immunogenic composition disclosed herein is administered in combination with an anti-inflammatory, e.g., a steroid or non-steroidal anti-inflammatory agents. Administration may be by any suitable mode known in the art. In a particular embodiment, administration is subcutaneous or intramuscular.

The useful dosage of the vaccine administered may vary. In one embodiment, the suitable dose is about 100 mcg (100 mcL) or more, more particularly, about 100 mcg, about 150 mcg, about 200 mcg, about 250 mcg, or about 300 mcg or more.

Dosage can be by a single dose schedule or a multiple dose schedule. Multiple doses may be used in a primary immunization schedule and/or in a booster immunization schedule. In a multiple dose schedule the various doses may be given by the same or different routes, e.g., a parenteral prime and mucosal boost, a mucosal prime and parenteral boost, etc. Multiple doses will typically be administered at least about 3 weeks apart, more particularly about 4 weeks apart.

The immunogenic formulation is provided, in certain embodiments, in unit dosage form (e.g., a vial) for ease of administration and uniformity of dosage. The unit dosage form may be provided, in certain embodiments, as a component of a kit that may optionally contain instructions for use.

The subject may be an animal, preferably a vertebrate, more preferably a mammal. Exemplary subject includes, e.g., a human, a cow, a pig, a chicken, a cat or a dog, as the infectious agents covered herein may be problematic across a wide range of species. Where the vaccine is for prophylactic use, the human is preferably a teenager, or an adult: where the vaccine is for therapeutic use, the human is preferably a teenager or an adult. In certain embodiment, the subject is a child.

In one embodiment, a method is disclosed a method of treating or preventing HIV infection in a subject comprising administering to the subject a therapeutically effective amount of the immunologic composition (e.g., vaccine) disclosed herein. In one embodiment, the HIV infection is at an acute stage. In one embodiment, the method further comprises administering to the subject another anti-viral agent.

### V. Methods of Preparation

The immunogenic compositions (e.g., vaccines) disclosed herein may be prepared by any suitable method.

The peptide of the immunogenic composition disclosed herein can be made in any suitable way, including purifying naturally occurring proteins, optionally proteolytically cleaving the proteins to obtain the desired functional domains, and conjugating the functional domains to other functional domains. Peptides can also be chemically synthesized, and optionally chemically conjugated to other peptides or chemical moieties.

Once a lipophilic phase is made, it is blended with an aqueous phase (e.g., water, saline, or any other aqueous solution which will be used to hydrate the lipids), which contains peptide antigens, under shear mixing conditions to form the adjuvanted peptide vaccine.

In one embodiment, the lipid vesicle (e.g., noisome) is prepared utilizing high sheer technology.

In one embodiment, the lipid vesicle (e.g., non-phospholipid based liposome) is loaded by a method selected from the group consisting of direct entrapment or remote loading.

The final concentration of peptide in the adjuvanted vaccine may vary. In one embodiment, the final concentration is 1.0 mg peptide in 1.0 g adjuvant, more particularly between about 0.8 mg/mL to about 1.0 mg/mL

In certain embodiments, the liposome is processed, e.g., by freeze drying, then reconstituted at use.

The following Examples will clearly illustrate the efficacy of the invention.

### EXAMPLES

### Example 1: Preparation of Peptides

A variety of peptides were prepared as described below.

A. Solid Phase Peptide Synthesis

In all cases the Solid Phase Peptide Synthesis (SPPS) was carried out using a CSBio H SPPS Instrument. All syntheses was conducted on Rink amide resin using standard Fmoc conditions. Rink amide resin was added to the reaction vessel, which was clamped in the SPPS instrument. HOBt and the relevant protected amino acids (see Table I for details) were added to glass AA reservoirs, which were loaded into the instrument in order, from the C-terminal amino acid to the N-terminal amino acid. Stock solutions of diisopropylcarbodiimide (DIC) (0.5 M) in DMF and piperidine (20%) in D1M1F were prepared and loaded into the instrument, solvent delivery was accomplished with 6 psi of N2. The resin was pre-swelled with DMF (3 washes over 10 min), followed by all coupling steps: piperidinemediated Fmoc-deprotection followed by DIC-mediated amide coupling, (all done at 60 deg. C., total time per step approx. 40 min.), and a final Fmoc-deprotection step of the N-terminus. In the case of the branched structure a Di-Fmoc Lysine was coupled at the end of each sequence which allowed multiple chains to be assembled after the Di-Fmoc was removed.

Once the synthesis was complete, the resin was collected on a filter funnel by vacuum filtration and washed with DCM (3×50 mL) to facilitate removal of residual DMF followed by MeOH (3×50 mL) to shrink the resin. The resin-bound protected peptide was then dried under vacuum and stored at ambient temperature.

B. Cleavage and Deprotection

A 20 ml scintillation vial was charged with a stir bar and resin-bound peptide was added. Separately, a cleavage cocktail was prepared by adding triflouroacetic acid (TFA) 95%, triisopropylsilane (TIS) 2.5%, and purified water 2.5% to a 15 mL plastic tube (for peptides with cysteines approx. 2% ethanedithiol (EDT) was added) and mixed well. The cleavage cocktail was added all at once to the scintillation vial. The vial was loosely capped (to allow escape of CO2) and the mixture was stirred for 3-4 hrs at ambient temperature. At this point, the solid resin byproducts were removed by vacuum filtration, and the filtered crude product precipitated in cold Et2O. The precipitate was spun down at approx. 3000 rpm, the ether removed by decanting and this process repeated two more times. The solid precipitate was dried under vacuum, ready for purification.

C. Purification

The peptides were purified by reverse phase HPLC. HPLC was performed on a C18 protein column using dilute aqueous TFA (0.1% TFA, 99.9% Milli-Q purified water v/v) and acetonitrile (ACN) as eluents. The solvent gradient increased from 0% ACN to 75% ACN over 35 min, then to 90% ACN over 5 min and held at 90% ACN for 5 min. The product fractions were frozen at −80° C. then lyophilized to form a white powder. The compounds were characterized for purity by analytical HPLC and for identity by mass spectrometry.

TABLE 1

Fmoc-Arg(Pbf)-OH
Fmoc-Asn(Trt)-OH
Fmoc-Cys(Trt)-OH
Fmoc-Glu(OtBu)—OH
Fmoc-Gln(Trt)-OH
Fmoc-Gly-OH
Fmoc-His(Trt)-OH
Fmoc-Leu-OH
Fmoc-Lys(Boc)—OH
Fmoc-Phe-OH
Fmoc-Pro-OH
Fmoc-Ser(tBu)—OH
Fmoc-Thr-(tBu)
Fmoc-Tyr(tBu)—OH
Fmoc-Val-OH
Fmoc-Lys-(Fmoc)—OH

### Example 2: Immunizations

Syrian golden hamsters were immunized on day 0, 28, 56, with approximately 250 uL of peptide in 250 uL of adjuvant with adjuvanted vaccines containing the peptides cngvegfnc (SEQ ID NO: 6), or ygfqptYgvgy (SEQ ID NO: 9), or a structure containing four copies of cngvegfnc (SEQ ID NO: 6) and three copies of ygfqptYgvgy (SEQ ID NO: 9). Groups of three animals were immunized subcutaneously or intramuscularly. Serum was collected and data from the day 27 and 55 bleeds are shown below. A nitrocellulose blot technique for peptides and proteins was developed and used in this study. The peptides were Peptide A(cngvegfnc (SEQ ID NO: 6)), the proteins were Receptor Binding Domain (amino acid number 319-541 of the Spike protein), and the S1 (amino acid number 16-635 of the Spike protein). These proteins were utilized to detect IgG antibodies in groups of three animals with the Vitamin E adjuvanted non-phospholipid-based liposome. The procedure is delineated in Example 3.

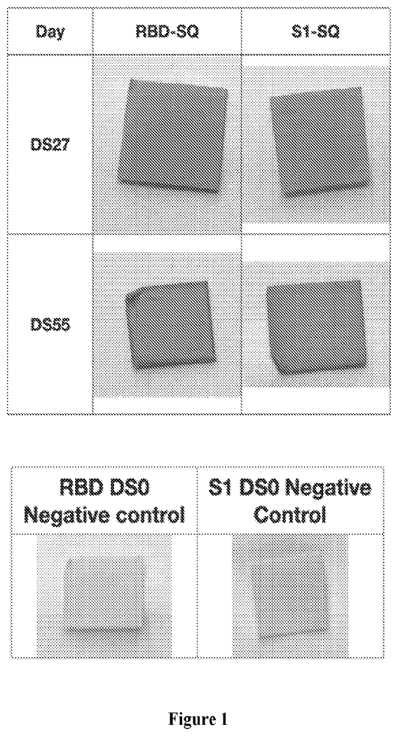

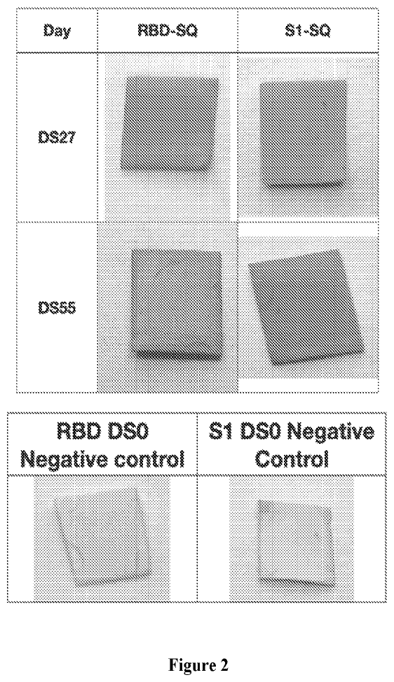

SVE are the initials for the Vitamin E containing non-phospholipid-based liposome adjuvant. SQ stands for subcutaneous administration of vaccines. FIG. 1 shows the day 27 and 55 bleed data. Blot images for IgG antibodies directed against the proteins Receptor Binding Domain and the S1 subunit of the Spike protein. In FIG. 1 the hamsters were immunized SQ or IM with SVE-Peptide A cngvegfnc (SEQ ID NO: 6).

The adjuvant formulations used for the above experiments were prepared using a reciprocating syringe technique which produced 5 milliliters of adjuvanted peptide.

The lipid formulation was composed of polyoxyethylene-2-stearyl-ether (40.0 g); cholesterol (17.0 g) Vitamin E (8.5 g), oleic acid (350 l). Peptides were solubilized in sterile water for injection at a concentration of 1.25 mg/mL. The lipid:diluent ratio on mixing was 1:4 on a volume basis. The final concentration of peptide in the adjuvanted vaccine was 1.0 mg peptide in 1.0 g adjuvant.

### Example 3: Methods to Detect IgG Antibodies Directed Against Peptides and Proteins Receptor Binding Domain and S1 in Hamster Serum

Nitrocellulose strips were cut into 0.5×0.5 cm squares and submerged in a 1 mL solution of peptide at 200 ug/mL or 0.25 μg/mL for RBD or S1 antigen diluted in PBS overnight at room temperature (RT).
Strips were placed in a 6 well dish and dried for 1 hr at RT
Strips were blocked in 1 mL Blocker buffer (1% milk protein in PBS) for 1 hr at RT on plate rocker.
Blocker was removed and 0.5 mL Blocker added to each well
Serum of pooled animal samples were added at a dilution of 1:20 to each well and incubated for 2 hours at RT on plate rocker.
Wells were washed 2× with 4 mL PBS for 4 minutes each. Secondary antibody conjugated to alkaline phosphatase was added in Blocker at a dilution of 1:50 to each well and incubated for 1 hr at RT on plate rocker.
Developing solution was made by adding 10 mg Naphthol AS_MX phosphate disodium salt and 22 mg Fast Red TR salt into 10 mL Tris-HCL pH=8.0.
Wells were washed 2× with 4 mL PBS for 4 minutes each and 1× with 4 mL Tris-HCL pH=8.0. Nitrocellulose was developed by the addition of 1 mL developing solution. Positive results appeared red when compared to negative control. Washed with 2 mL PBS.

SEQUENCE LISTINGS

| SEQUENCE ID NO: 1 | |
|---|---|
| LOCUS QJG65958 1273 aa linear VRL 30-APR-2020 | |
| DEFINITION | surface glycoprotein [Severe acute respiratory syndrome 2]. |
| coronavirus | |
| ACCESSION | QJG65958 |
| VERSION | QJG65958.1 |
| DBSOURCE | accession MT415377.1 |
| KEYWORDS | . |
| SOURCE | Severe acute respiratory syndrome coronavirus 2 (SARS-COV-2) |
| ORGANISM | Severe acute respiratory syndrome coronavirus 2 |
| | Viruses; Riboviria; Orthornavirae; Pisuviricota; Pisoniviricetes; |
| | Nidovirales; Cornidovirineae; Coronaviridae; Orthocoronavirinae; |
| | Betacoronavirus; Sarbecovirus. |
| REFERENCE | 1 (residues 1 to 1273) |
| AUTHORS | Yuan, Y., He, J., Gong, L., Li, W., Jiang, L., Liu, J., Chen, Q., Yu, J., |
| | Hou, S., Shi, Y., Lu, S., Zhang, Z., Ge, Y., Sa, N., He, L., Wu, J., |
| | Sun, Y. and Liu, Z. |
| TITLE | Molecular epidemiology of SARS-Cov-2 clusters caused by |
| JOURNAL | asymptomatic cases in Anhui Province, china |
| | Unpublished |
| REFERENCE | 2 (residues 1 to 1273) |
| AUTHORS | Yuan, Y., He, J., Gong, L., Li, W., Jiang, L., Liu, J., Chen, Q., Yu, J., |
| | Hou, S., Shi, Y., Lu, S., Zhang, Z., Ge, Y., Sa, N., He, L., Wu, J., |
| | Sun, Y.and Liu, Z. |
| TITLE | Direct Submission |
| JOURNAL | Submitted (28-APR-2020) Anhui Provincial Center for Disease |
| Control | and Prevention, 12560, Fanhua Avenue, Anhui Province, China |
| COMMENT | ##Assembly-Data-START## |
| | Sequencing Technology :: Sanger dideoxy sequencing |
| | ##Assembly-Data-END## |
| FEATURES | Location/Qualifiers |
| source | 1 . . . 1273 |
| | /organism = “Severe acute respiratory syndrome coronavirus 2” |
| | /isolate = “SARS-COV-2/human/CHN/MAS635/2020” |
| | /host = “Homo sapiens” |
| | /db_xref = “taxon:2697049” |
| | /country = “China” |
| | /collection_date = “2020-02-18” |
| Protein | 1 . . . 1273 |
| | /product = “surface glycoprotein” |
| Region | 13 . . . 304 |
| | /region name = “SARS-Cov-like Spike S1 NTD” |
| | /note = “N-terminal domain of the S1 subunit of the Spike |
| | (S) protein from Severe acute respiratory syndrome |
| | coronavirus and related betacoronaviruses in the B |
| | lineage; cd21624” |
| | /db_xref = “CDD:394950” |
| Site | order (38, 41 . . . 44, 113, 115, 167, 198 . . . 200, 225, 228, 230 . . . 234, |
| | 281 . . . 283) |
| | /site_type = “other” |
| | /note = “trimer interface [polypeptide binding]” |
| | /db_xref = “CDD:394950” |
| Region | 319 . . . 541 |
| | /region_name = “SARS-CoV-2_Sp i ke_S1_RBD” |
| | /note = “receptor-binding domain of the S1 subunit of |
| | severe |
| | acute respiratory syndrome coronavirus 2 Spike (S) |
| | protein; cd21480” |
| | /db_xref = “CDD:394827” |
| Site | order (319, 321, 357, 381 . . . 383, 386, 390, 394, 396, 413, 516 . . . 517, |
| | 521) |
| | /site_type = “other” |
| | /note = “trimer interface [polypeptide binding]” |
| | /db_xref = “CDD:394827” |
| Site | order (369 . . . 370, 372, 374, 376 . . . 378, 380, 382, |
| | 384 . . . 386, 389 . . . 390, 392, 428 . . . 430, 515, 517, 519) |
| | /site_type = “other” |
| | /note = “cryptic epitope [polypeptide binding]” |
| | /db_xref = “CDD:394827” |
| | order (417, 446, 449, 453, 455 . . . 456, 475, 486 . . . 487, 489, 493, 496, |
| Site | 498, 500 . . . 502, 505) |
| | /site_type = “other” |
| | /note = “receptor binding site [polypeptide binding]” |
| | /db_xref = “CDD:394827” |
| Site | 438 . . . 508 |
| | /site_type = “other” |
| | /note = “receptor binding motif” |
| | /db_xref = “CDD:394827” |
| Region | 662 . . . 1270 |
| | /region_name = “Corona S2” |
| | /note = “Coronavirus S2 glycoprotein; pfam01601” |
| | /db_xref = “CDD:279881” |
| CDS | 1 . . . 1273 |
| | /gene = “S” |
| | /coded_by = “MT415377.1:1 . . . 3822” |

```
ORIGIN
      1 mfvflvllpl vssqcvnltt rtqlppaytn sftrgvyypd kvfrssvlhs tqdlflpffs
    61 nvtwfhaihv sgtngtkrfd npvlpfndgv yfasteksni irgwifgttl dsktqslliv
  121 nnatnvvikv cefqfcndpf lgvyyhknnk swmesefrvy ssannctfey vsqpflmdle
  181 gkqgnfknlr efvfknidgy fkiyskhtpi nlvrdlpqgf saleplvdlp iginitrfqt
  241 llalhrsylt pgdsssgwta gaaayyvgyl qpmtyilkyn engtitdavd caldplsetk
  301 ctlksftvek giyqtsnfrv qptesivrfp nitnlcpfge vfnatrfasv yawnrkrisn
  361 cvadysvlyn sasfstfkcy gvsptklndl cftnvyadsf virgdevrqi apgqtgkiad
  421 ynyklpddft gcviawnsnn ldskvggnyn ylyrlfrksn lkpferdist eiyqagstpc
  481 ngvegfncyf plqsygfqpt ngvgyqpyrv vvlsfellha patvcgpkks tnlvknkcvn
  541 fnfngltgtg vltesnkkfl pfqqfgrdia dttdavrdpq tleilditpc sfggvsvitp
  601 gtntsnqvav lyqdvnctev pvaihadqlt ptwrvystgs nvfqtragcl igaehvnnsy
  661 ecdipigagi casyqtqtns prrarsvasq siiaytmslg aensvaysnn siaiptnfti
  721 svtteilpvs mtktsvdctm yicgdsteci nlllqygsfc tqlnraltgi aveqdkntqe
  781 vfaqvkqiyk tppikdfggf nfsqilpdps kpskrsfied llfnkvtlad agfikqygdc
  841 lgdiaardli caqkfngltv lpplltdemi aqytsallag titsgwtfga gaalqipfam
  901 qmayrfngig vtqnvlyenq klianqfnsa igkiqdslss tasalgklqd vvnqnaqaln
  961 tlvkqlssnf gaissvlndi lsrldkveae vqidrlitgr lqslqtyvtq qliraaeira
1021 sanlaatkms ecvlgqskrv dfcgkgyhlm sfpqsaphgv vflhvtyvpa qeknfttapa
1081 ichdgkahfp regvfvsngt hwfvtqrnfy epqiittdnt fvsgncdvvi givnntvydp
1141 1qpeldsfke eldkyfknht spdvdlgdis ginasvvniq keidrlneva knlneslidl
1201 gelgkyegyi kwpwyiwlgf iagliaivmv timlccmtsc csclkgccsc gscckfdedd
1261 sepvlkgvkl hyt
SEQUENCE ID. NO.: 2
United Kingdom (B.1.1.7) Single Mutation in Receptor Binding Motif SARS CoV-2
Spike protein sequence:
UK ORIGIN
      1 mfvflvllpl vs [S1 NTD aa13 [sqcvnltt([rtqlppa ytnsftr])gvyypd kvfrssvlhs tqdlflpffs
    61 nvtwfhaihv(D) sgtngtkrfd npvlpfndgv yfasteksni irgwifgttl dsktqslliv
  121 nnatnwikv cefqfcndpf lgvyy(D)hknnk swmesefrvy ssannctfey vsqpflmdle
  181 gkqgnfknlr efvfknidgy fkiyskhtpi nlvrdlpqgf saleplvdlp iginitrfqt
  241 llalhrsylt pgdsssgwta gaaayyvgyl qprtfllkyn engtitdavd caldplsetk
  301 ctlk]S1 NTD aa304] sftvek giyqtsnf{S1 PBD aa319{rv qptesivrfp nitnlcpfge vfnatrfasv yawnrkrisn
  361 cvadysvlyn sasfstfkcy gvsptklndl cftnvyadsf virgdevrqi apgq ([tgkiad
  421 ynyklpddft g]) cviawn{S1 RBM aa438{snn ldskvggnyn ylyrlfrksn lkpferdist eiyqagstp([c
  481 ngvegfnc]yf plqs[ygfqpt n(Ysubn) gvgy]) qpy{S1 RBM aa508{rvvvlsfellha patvcgpkks tnlvknkcvn
  541 f}S1 RBD aa541}nfngltgtg vliesnkkfl pfqqfgrdia(Dsuba) dttdavrdpq tleilditpc sfggvsvitp
  601 gtntsnqvav lyqyvnctev pvaihadqlt ptwrvystgs nvfqtragcl igaehvnnsy
  661 e[S2 aa662[cdipigagi casyqtqtns {p(Hsubp)rrar}svasq siiaytmslg aensvaysnn siaipt(Isubt) nfti
  721 svtteilpvs mtktsvdctm yicgdstecs nlllqygsfc tqlnraltgi aveqdkntqe
  781 vfaqvkqiyk tppikdfggf nfsqilpdps kpskrsfied llfnkvtlad agfikqygdc
  841 lgdiaardli caqkfngltv lpplltdemiaqyt sall([agtitsgwtiiga]) gaalqipfam
  901 qmayrfngig vtqnvlyenq klianqfnsa igkiqdslss tasalgklqd wnqnaqaln
  961 tlvkqlssnf gaissvlndi Is(Asnibs)rldkveae vqidrlitgr lqslqtyvtq qliraaeira
1021 sanlaatkms ecvlgqskrv dfcgkgyhlm sfpqsaphgv vflhvtyvpa qeknfttapa
1081 ichdgkahfp regvfvsngt hwfvtqrnfy epqiittd(Asubd)([ntfvsgncdvvi givnnt])(vydp
1141 lqpeldsfke eldkyfknht spdvdlgdis ginaswniq keidrlneva knlneslidl
1201 qelgkyeqyi kwpwyiwlgf agliaivmv timlccmtsc csclkgccsc gscckfdedd
1261 sepv1kgvkl] S2 aa1270]hyt}
SEQUENCE ID NO.: 3
United Kingdom(B.1.1.7) Double Mutation in Receptor Binding Motif SARS CoV-2
Spike protein sequence:
UK ORIGIN
    1 mfvflvllpl vs[S1 NTD aa13[sqcvnltt([rtqlppa ytnsftr])gvyypd kvfrssvlhs tqdlflpffs
  61 nvtwfhaihv(D) sgtngtkrfd npvlpfndgv yfasteksni irgwifgttl dsktqslliv
  121 nnatnwikv cefqfcndpf lgvyy(D)hknnk swmesefrvy ssannctfey vsqpflmdle
  181 gkqgnfknlr efvfknidgy fkiyskhtpi nlvrdlpqgf saleplvdlp iginitrfqt
  241 llalhrsylt pgdsssgwta gaaayyvgyl qprtfllkyn engtitdavd caldplsetk
  301 ctlk]S1 NTD aa304]sftvek giyqtsnf{S1 RBD aa319{rv qptesivrfp nitnlcpfge vfnatrfasv yawnrkrisn
  361 cvadysvlyn sasfstfkcy gvsptklndl cftnvyadsf virgdevrqi apgq ([tgkiad
  421 ynyklpddft g])cviawn{S1 RBM aa438{snn ldskvggnyn ylyrlfrksn lkpferdist eiyqagstp([c
  481 ngve(Ksube)gfnc]yf plqs[ygfqpt n(Ysubn)gvgy])qpy{S1 RBM aa508{rv wlsfellha patvcgpkks
          tnlvknkcvn
  541 f}S1 RBD aa541}nfngltgtg vliesnkkfl pfqqfgrdia(Dsuba) dttdavrdpq tleilditpc sfggvsvitp
  601 gtntsnqvav lyqgvnctev pvaihadqlt ptwrvystgs nvfqtragcl igaehvnnsy
  661 e[S2 aa662[cdipigagi casyqtqtns {p(Hsubp)rrarjsvasq siiaytmslg aensvaysnn siaipt(Isubt)nfti
  721 svtteilpvs mtktsvdctm yicgdstecs nlllqygsfc tqlnraltgi aveqdkntqe
  781 vfaqvkqiyk tppikdfggf nfsqilpdps kpskrsfied llfnkvtl ad agfikqygdc
  841 lgdiaardli caqkfngltv lpplltdemiaqyt sail([agtitsgwtfga]) gaalqipfam
  901 qmayrfngig vtqnvlyenq klianqfnsa igkiqdslss tasalgklqd vvnqnaqaln
  961 tlvkqlssnf gaissvlndi ls(Asubs)rldkveae vqidrlitgr lqslqtyvtq qliraaeira
1021 sanlaatkms ecvlgqskrv dfcgkgyhlm sfpqsaphgv vflhvtyvpa qeknfttapa
1081 ichdgkahfp regvfvsngt hwfvtqrnfy epqiittd(Asubd)([nt fvsgncdwi givnnt]) (vydp
1141 lqpeldsfke eldkyfknht spdvdlgdis ginasvvniq keidrlneva knlneslidl
1201 qelqkyeqyi kwpwyiwlqf iaqliaivmv timlccmtsc csclkqccsc qscckfdedd
1261 sepvlkgvkl]S2 aa1270]hyt)
SEQUENCE ID NO.: 4
South Africa (B.1.351) Double Mutation in Receptor Binding Motif SARS CoV-2
Spike protein sequence:
SA ORIGIN
      1 mfvflvllpl vs[S1 NTD aa13[sqcvnl(Fsubl)tt ([rtqlppa ytnsftr]) rgvyypd kvfrssvlhs tqdlflpffs
    61 nvtwfhaihv sgtngtkrfd(Asubd) npvlpfndgv yfasteksni irgwifgttl dsktqslliv
  121 nnatnvvikv cefqfcndpf lgvyyhknnk swmesefrvy ssannctfey vsqpflmdle
  181 gkqgnfknlr efvfknidgy fkiyskhtpi ylvrd(Gsubd)lpqgf saleplvdlp iginitrfqt
  241 llal(D) hr(Isubr)sylt pgdsssgwta gaaayyvgyl qprtfllkyn engtitdavd caldplsetk
  301 ctlk]S1 NTD aa304] sftvek giyqtsnfrv qptesivrf{S1 RBD aa319{p nitnlcpfge vfnatrfasv yawnrkrisn
  361 cvadysvlyn sasfstfkcy gvsptklndl cftnvyadsf virgdevrqi apgq([tgk(Nsubk)iad
  421 ynyklpddft g]) cviawn{S1 RBM aa438{snn ldskvggnyn ylyrlfrksn Ikpferdist eiygagstp([c
  481 ngve(Ksube)gfnc] yfplqs[ygfqpt n(Ysubn)gvgy]}qpy} S1 RBM aa508} rvvvlsfellha
          patvcgpkks tnlvknkcvn
  541 f}S1 RBD aa541} nfngltgtg vltesnkkfl pfqqfgrdia dttdavrdpq tleilditpc sfggvsvitp
  601 gtntsnqvav lyqgvnctev pvaihadqlt ptwrvystgs nvfqtragcl igaehvnnsy
  661 ec [S2 aa662 [dipigagi casyqtqtns {prrar} svasq sii aytmslga(Vsuba) ensvaysnn siaiptnfti
  721 svtteilpvs mtktsvdctm yicgdst ecs nlllqygsfc tqlnraltgi aveqdkntqe
  781 vfaqvkqiyk tppikdfggf nfsqilpdps kpskrsfied llfnkvtlad agfikqygdc
  841 1gdiaardli caqkfngltv lpplltdemi aqytsall([agtitsgwtfga])gaalqipfam
  901 qmayrfngig vtqnvlyenq klianqfnsa igkiqdslss tasalgklqd vvnqnaqaln
  961 tlvkqlssnf gaissvIndi 1srldkveae vqidrlitgr lqslqtyvtq qliraaeira
1021 sanlaatkms ecvlgqskrv dfcgkgyhlm sfpqsaphgv vflhvtyvpa qeknfttapa
1081 ichdgkahfp regvfvsngt hwfvtqrnfy epqiittd ([nt fvsgncdvvi givnnt])(vydp
1141 lqpeldsfke eldkyfknht spdvdlgdis ginasvvniq keidrlneva knlneslidl
1201 qelgkyegyi kwpwyiwlgf iagliaivmv timlccmtsc csclkgccsc gscckfdedd
1261 sepvlkgvk1] S2 aa1270] hyt)
SEQUENCE ID. NO.: 5
Brazil (B.1.1.248) Double Mutation in Receptor Binding Motif SARS CoV-2 Spike
protein sequence:
Brazil ORIGIN
    1 mfvflvllpl vs [S1 NTD aa13 [sqcvnl (Fsubl)t
          t (Nsubt) ([rtqlp (Ssubp) payt nsftr]) rgvyypd kvfrssvlhs tqdlflpffs
    61 nvtwfhaihv sgtngtkrfd npvlpfndgv yfasteksni irgwifgttl dsktqslliv
  121 nnatnvvikv cefqfond (Ysubd) pf lgvyyhknnk swmesefrvy ssannctfey vsqpflmdle
  181 gkqgnfknlr (Ssubr) efvfknidgy fkiyskhtpi ylvrdlpqgf saleplvdlp iginitrfqt
  241 llalhrsylt pgdsssgwta gaaayyvgyl qprtfllkyn engtitdavd caldplsetk
  301 ctlk] S1 NTD aa304] sftvek giyqtsnfrv qptesivrf {S1 RBD aa319{p nitnlcpfge
          vfnatrfasv yawnrkrisn
  361 cvadysvlyn sasfstfkcy gvsptklndl cftnvyadsf virgdevrqi apgq
          ([tgk(Tsubk) iad
  421 ynyklpddft g]) cviawn {S1 RBM aa438{snn ldskvggnyn ylyrlfrksn Ikpferdist eiyqagstp ([c
  481 ngve (Ksube) gfnc] yfplqs [ygfqpt n(Ysubn)gvgy]) qpy} S1 RBM aa508}
          rvvvlsfellha patvcgpkks tnlvknkcvn
  541 f}S1 RBD aa541} nfngltgtg vltesnkkfl pfqqfgrdia dttdavrdpq tleilditpc sfggvsvitp
  601 gtntsnqvav lyqgvnctev pvaihadqlt ptwrvystgs nvfqtragcl igaeh (ysubh) vnnsy
  661 ec [S2 aa662 [dipigagi casyqtqtns prrarsvasq siiaytmslg aensvaysnn siaiptnfti
  721 svtteilpvs mtktsvdctm yicgdstecs nlllqygsfc tqlnraltgi aveqdkntqe
  781 vfaqvkqiyk tppikdfggf nfsqilpdps kpskrsfied llfnkvtlad agfikqygdc
  841 1gdiaardli caqkfngltv lpplltdemi aqytsall([agtitsgwtfga]) gaalqipfam
  901 qmayrfngig vtqnvlyenq klianqfnsa igkiqdslss tasalgklqd vvngnagaln
  961 tlvkqlssnf gaissvIndi 1srldkveae vqidrlitgr lqslqtyvtq qliraaeira
1021 sanlaat (Isubt) kms ecvlgqskrv dfcgkgyhlm sfpqsaphgv vflhvtyvpa qeknfttapa
1081 ichdgkahfp regvfvsngt hwfvtqrnfy epqiittd([nt fvsgncdvvi givnnt]) (vydp
1141 lqpeldsfke eldkyfknht spdvdlgdis ginasv (Fsubv) vniq keidrlneva knlneslidl
1201 qelgkyegyi kwpwyiwlgf iagliaivmv timlccmtsc csclkgccsc gscckfdedd
1261 sepvlkgvk1]S2 aa1270] hyt)
```

1. A vaccine comprising at least one peptide and a non-phospholipid liposome, wherein the liposome comprises Vitamin E.

2. The vaccine of claim 1, wherein the liposome comprises between 2-10 bilayers surrounding an amorphous central cavity, and wherein said non-phospholipids are selected from the group consisting of polyoxyethylene fatty acid esters, polyoxyethylene fatty acid ethers, polyoxyethylene sorbitan esters, polyoxyethylene glyceryl mono- and diesters, glyceryl mono- and distearate, sucrose distearate, propylene glycol stearate, long chain acyl hexosamides, long chain acyl amino acid amides, long chain acyl amides, glyceryl mono- and diesters, dimethyl acyl amines, C 12-C 20 fatty alcohols, C 12-C 20 glycol monoesters, and C 12-C 20 fatty acids.

3. The vaccine of claim 1, wherein the at least one peptide is encapsulated within the liposome.

4. The vaccine of claim 1, wherein said peptide is encapsulated within the amorphous central cavity of the liposome.

5. The vaccine of claim 1, wherein the at least one peptide is selected from the group consisting of peptide A (cngvegfnc), peptide B (ygfqptngvgy), and peptide D (construct containing four copies of Peptide A and three copies of Peptide B).

6. The vaccine of claim 1, wherein the at least one peptide is selected from the group consisting of Peptide A (cngvegfnc), Peptide B (ygfqptngvgy), and Peptide D (construct which contains four copies of Peptide A and three copies of Peptide B).

7. The vaccine of claim 1, wherein the non-phospholipid liposome further comprises at least one sterol selected from the group consisting of cholesterol and cholesterol derivatives.

8. The vaccine of claim 1, wherein the non-phospholipid liposome comprises an amorphous central cavity containing Vitamin E.

9. The vaccine of claim 1, wherein the vaccine generates antibodies that recognize the Receptor Binding Domain (RBD) and the SI protein of SARS-CoV-2.

9. A vaccine comprising a peptide antigen and a non-phospholipid liposome, wherein the peptide antigen comprises seven peptides selected from the group consisting of: cngvegfnc, ygfqptngvgy, cngvKgfnc, ygfqptYgvgy and combinations thereof, wherein the vaccine generates antibodies that recognize the Receptor Binding Domain, and SI protein of SARS-CoV-2.

10. A vaccine comprising a peptide antigen and a non-phospholipid liposome, wherein the peptide antigen comprises seven peptides selected from the group consisting of cngvegfnc, ygfqptngvgy, cngvKgfnc, ygfqptYgvgy and combinations thereof, wherein the vaccine generates antibodies that recognizes the Receptor Binding Domain, and SI protein of SARS-CoV-2.

11. The vaccine of claim 1, wherein the at least one peptide is derived from a COVID-19 isolate selected from the group consisting of the Wuhan isolates, the United Kingdom Isolates, the South Africa isolates, the Brazilian Isolate, and the Cal-20C isolate.

12. The vaccine of claim 1, wherein the at least one peptide is selected from the group of peptides consisting of amino acids 434-444, amino acids 449-460, amino acids 480-488, amino acids 495-505, amino acids 662-671, or amino acids 679-688, in each case of SEQ ID. No.: 1.
